(19) United States
(12) Patent Application Publication
(10) Pub. No.: US 2004/0067985 A1
(43) Pub. Date: Apr. 8, 2004

(54) Method of inhibiting angiogenesis

(76) Inventors: Fortuna Haviv, Deerfield, IL (US); Michael F. Bradley, Wadsworth, IL (US); Jurgen Dinges, Grayslake, IL (US); Daryl R. Sauer, Trevor, WI (US); Jack Henkin, Highland Park, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/264,421

(22) Filed: Oct. 4, 2002

(51) Int. Cl. A61K 31/455; C07D 213/56

(52) U.S. Cl. 514/355; 546/316

(57) ABSTRACT

Compounds having the formula

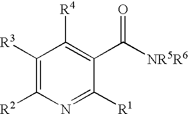

are angiogenesis inhibitors. Also disclosed are compositions containing the compounds, methods of making the compounds, and methods of treatment using the compounds.

### TECHNICAL FIELD

The present invention relates to methods of inhibiting angiogenesis, methods of treating cancer, and compounds having activity useful for treating conditions which arise from or are exacerbated by angiogenesis. Also disclosed are pharmaceutical compositions comprising the compounds and methods of treatment using the compounds.

### BACKGROUND OF THE INVENTION

Angiogenesis is the fundamental process by which new blood vessels are formed and is essential to a variety of normal body activities (such as reproduction, development and wound repair). Although the process is not completely understood, it is believed to involve a complex interplay of molecules which both stimulate and inhibit the growth of endothelial cells, the primary cells of the capillary blood vessels. Under normal conditions these molecules appear to maintain the microvasculature in a quiescent state (i.e., one of no capillary growth) for prolonged periods that may last for weeks, or in some cases, decades. However, when necessary, such as during wound repair, these same cells can undergo rapid proliferation and turnover within as little as five days.

Although angiogenesis is a highly regulated process under normal conditions, many diseases (characterized as “angiogenic diseases”) are driven by persistent unregulated angiogenesis. Otherwise stated, unregulated angiogenesis may either cause a particular disease directly or exacerbate an existing pathological condition. For example, the growth and metastasis of solid tumors have been shown to be angiogenesis-dependent. Based on these findings, there is a continuing need for compounds which demonstrate antiangiogenic activity due to their potential use in the treatment of various diseases such as cancer.

### SUMMARY OF THE INVENTION

In its principle embodiment, the present invention provides a method of inhibiting angiogenesis comprising administering to a mammal in need of such treatment a therapeutically effective amount of a compound of formula (I)

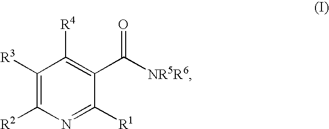

or a therapeutically salt thereof, wherein

R¹, R², R³, and R⁴ are independently selected from the group consisting of hydrogen, alkoxy, alkoxycarbonylalkyl, alkyl, amino, aryl, arylalkyl, cyano, cyanoalkyl, cycloalkyl, (cycloalkyl)alkyl, halo, haloalkoxy, haloalkyl, heteroaryl, heteroarylalkyl, heterocycle, (heterocycle)alkyl, hydroxy, hydroxyalkyl, and nitroalkyl; and

R⁵ and R⁶ are independently selected from the group consisting of hydrogen, alkoxyalkyl, alkyl, alkynyl, alkylsulfanylalkyl, aminoalkyl, cyanoalkyl, cycloalkyl, (cycloalkyl)alkyl, haloalkyl, heteroarylalkyl, and (heterocycle)alkyl.

In a preferred embodiment, the present invention provides a method of inhibiting angiogenesis comprising administering to a mammal in need of such treatment a therapeutically effective amount of a compound of formula (I) wherein

R¹, R², and R⁴ are hydrogen; and

R³ is other than hydrogen.

In another preferred embodiment, the present invention provides a method of inhibiting angiogenesis comprising administering to a mammal in need of such treatment a therapeutically effective amount of a compound of formula (I), or a therapeutically acceptable salt thereof, wherein

R¹, R³, and R⁴ are hydrogen;

R is other than hydrogen; and

R⁵ and R⁶ are alkyl.

In another preferred embodiment, the present invention provides a method of inhibiting angiogenesis comprising administering to a mammal in need of such treatment a therapeutically effective amount of a compound of formula (I), or a therapeutically acceptable salt thereof, wherein

R¹, R³, and R⁴ are hydrogen;

R is other than hydrogen; and

one of R⁵ and R⁶ is hydrogen and the other is alkyl.

In another preferred embodiment, the present invention provides a method of inhibiting angiogenesis comprising administering to a mammal in need of such treatment a therapeutically effective amount of a compound of formula (I), or a therapeutically acceptable salt thereof, wherein

R¹, R³, and R⁴ are hydrogen;

R² is other than hydrogen; and

one of R⁵ and R⁶ is selected from the group consisting of hydrogen and alkyl and the other is selected from the group consisting of cycloalkyl and (cycloalkyl)alkyl.

In another preferred embodiment, the present invention provides a method of inhibiting angiogenesis comprising administering to a mammal in need of such treatment a therapeutically effective amount of a compound of formula (I), or a therapeutically acceptable salt thereof, wherein

R¹, R³, and R⁴ are hydrogen;

R² is other than hydrogen; and

one of R⁵ and R⁶ is selected from the group consisting of hydrogen and alkyl and the other is selected from the group consisting of hydrogen, alkoxyalkyl, cyanoalkyl, haloalkyl, and (heterocycle)alkyl.

In another preferred embodiment, the present invention provides a method of inhibiting angiogenesis comprising administering to a mammal in need of such treatment a therapeutically effective amount of a compound of formula (I), or a therapeutically acceptable salt thereof, wherein

R¹, R³, and R⁴ are hydrogen;

R² is other than hydrogen; and

one of R⁵ and R⁶ is alkyl and the other is aminoalkyl.

In another preferred embodiment, the present invention provides a method of inhibiting angiogenesis comprising administering to a mammal in need of such treatment a therapeutically effective amount of a compound of formula (I), or a therapeutically acceptable salt thereof, wherein

R¹ is as defined for formula (I); and

R², R³, and R⁴ are hydrogen.

In another preferred embodiment, the present invention provides a method of inhibiting angiogenesis comprising administering to a mammal in need of such treatment a therapeutically effective amount of a compound of formula (I), or a therapeutically acceptable salt thereof, wherein

R¹ and R² are other than hydrogen;

R³ and R⁴ are hydrogen; and

one of R⁵ and R⁶ is alkyl and the other is selected from the group consisting of hydrogen and alkyl.

In another preferred embodiment, the present invention provides a method of inhibiting angiogenesis comprising administering to a mammal in need of such treatment a therapeutically effective amount of a compound of formula (I), or a therapeutically acceptable salt thereof, wherein

R¹ and R² are other than hydrogen;

R³ and R⁴ are hydrogen; and

one of R⁵ and R⁶ is selected from the group consisting of hydrogen and alkyl and the other is selected from the group consisting of alkoxyalkyl, cyanoalkyl and cycloalkyl.

In another preferred embodiment, the present invention provides a method of inhibiting angiogenesis comprising administering to a mammal in need of such treatment a therapeutically effective amount of a compound of formula (I), or a therapeutically acceptable salt thereof, wherein

R¹ and R² are other than hydrogen;

R³ and R⁴ are hydrogen; and

one of R⁵ and R⁶ is selected from the group consisting of hydrogen and alkyl and the other is selected from the group consisting of alkylsulfanylalkyl, alkynyl, (cycloalkyl)alkyl, and (heterocycle)alkyl.

In another embodiment the present invention provides a method of inhibiting angiogenesis comprising administering to a human in need of such treatment a therapeutically effective amount of a compound of formula (I) or a therapeutically acceptable salt thereof.

In another embodiment the present invention provides a method of treating cancer comprising administering to a mammal in need of such treatment a therapeutically effective amount of a compound of formula (I) or a therapeutically acceptable salt thereof.

In another embodiment the present invention provides a method of treating cancer comprising administering to a human in need of such treatment a therapeutically effective amount of a compound of formula (I) or a therapeutically acceptable salt thereof.

In another embodiment the present invention provides a compound of formula (II)

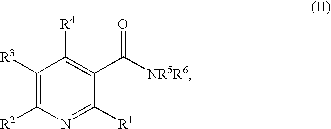

or a therapeutically acceptable salt thereof, wherein

R¹ and R⁴ are independently selected from the group consisting of hydrogen, alkoxy, alkoxycarbonylalkyl, alkyl, arylalkyl, cyano, cyanoalkyl, cycloalkyl, (cycloalkyl)alkyl, halo, haloalkoxy, haloalkyl, heteroaryl, heteroarylalkyl, heterocycle, (heterocycle)alkyl, hydroxy, hydroxyalkyl, and nitroalkyl;

R² and R³ are independently selected from the group consisting of hydrogen, alkoxy, alkoxycarbonylalkyl, alkyl, aryl, arylalkyl, cyano, cyanoalkyl, cycloalkyl, (cycloalkyl)alkyl, halo, haloalkoxy, haloalkyl, heteroaryl, heteroarylalkyl, heterocycle, (heterocycle)alkyl, hydroxy, and hydroxyalkyl; provided that at least one of R¹, R², R³, and R⁴ is other than hydrogen; and

one of R⁵ and R⁶ is alkyl and the other is selected from the group consisting of alkoxyalkyl and dialkylaminoalkyl.

In another embodiment the present invention provides a pharmaceutical composition comprising a compound of formula (II), or a therapeutically acceptable salt thereof, in combination with a therapeutically acceptable carrier.

In another embodiment the present invention provides a method of inhibiting angiogenesis comprising administering to a mammal in need of such treatment a therapeutically effective amount of a compound of formula (II) or a therapeutically acceptable salt thereof.

In another embodiment the present invention provides a method of inhibiting angiogenesis comprising administering to a human in need of such treatment a therapeutically effective amount of a compound of formula (II) or a therapeutically acceptable salt thereof.

In another embodiment the present invention provides a method of treating cancer comprising administering to a mammal in need of such treatment a therapeutically effective amount of a compound of formula (II) or a therapeutically acceptable salt thereof.

In another embodiment the present invention provides a method of treating cancer comprising administering to a human in need of such treatment a therapeutically effective amount of a compound of formula (II) or a therapeutically acceptable salt thereof.

### DETAILED DESCRIPTION OF THE INVENTION

As used in the present specification the following terms have the meanings indicated:

The term “alkoxy,” as used herein, refers to an alkyl group attached to the parent molecular moiety through an oxygen atom.

The term “alkoxyalkyl,” as used herein, refers to an alkyl group substituted by at least one alkoxy group.

The term “alkoxycarbonyl,” as used herein, refers to an alkoxy group attached to the parent molecular moiety through a carbonyl group.

The term “alkoxycarbonylalkyl,” as used herein, refers to an alkoxycarbonyl group attached to the parent molecular moiety through an alkyl group.

The term “alkyl,” as used herein, refers to a group derived from a straight or branched chain saturated hydrocarbon containing from one to ten carbon atoms.

The term “alkylcarbonyl,” as used herein, refers to an alkyl group attached to the parent molecular moiety through a carbonyl group.

The term “alkylsulfanyl,” as used herein, refers to an alkyl group attached to the parent molecular moiety through a sulfur atom.

The term “alkylsulfanylalkyl,” as used herein, refers to an alkylsulfanyl group attached to the parent molecular moiety through an alkyl group.

The term “alkynyl,” as used herein, refers to a straight or branched chain hydrocarbon of two to six carbon atoms containing at least one carbon-carbon triple bond.

The term “amino,” as used herein, refers to —NR^{a}R^{b}, wherein R^{a} and R^{b} are independently selected from the group consisting of hydrogen, alkyl, alkylcarbonyl, cycloalkyl, (cycloalkyl)alkyl, and unsubstituted phenyl.

The term “aminoalkyl,” as used herein, refers to an alkyl group substituted by at least one amino group.

The term “aminocarbonyl,” as used herein, refers to an amino group attached to the parent molecular moiety through a carbonyl group.

The term “aryl,” as used herein, refers to a phenyl group, or a bicyclic or tricyclic fused ring system wherein one or more of the fused rings is a phenyl group. Bicyclic fused ring systems are exemplified by a phenyl group fused to a monocyclic cycloalkenyl group, as defined herein, a monocyclic cycloalkyl group, as defined herein, or another phenyl group. Tricyclic fused ring systems are exemplified by a bicyclic fused ring system fused to a monocyclic cycloalkenyl group, as defined herein, a monocyclic cycloalkyl group, as defined herein, or another phenyl group. Representative examples of aryl include, but are not limited to, anthracenyl, azulenyl, fluorenyl, indanyl, indenyl, naphthyl, phenyl, and tetrahydronaphthyl. The aryl groups of the present invention can be optionally substituted with one, two, three, four, or five substituents independently selected from the group consisting of alkoxy, alkoxyalkyl, alkoxycarbonyl, alkyl, alkylcarbonyl, alkylsulfanyl, amino, aminoalkyl, aminocarbonyl, cyano, cyanoalkyl, halo, haloalkoxy, haloalkyl, nitro, and oxo.

The term “arylalkyl,” as used herein, refers to an aryl group attached to the parent molecular moiety through an alkyl group.

The term “carbonyl,” as used herein, refers to —C(O)—.

The term “cyano,” as used herein, refers to —CN.

The term “cyanoalkyl,” as used herein, refers to an alkyl group substituted with at least one cyano group.

The term “cycloalkenyl,” as used herein, refers to a non-aromatic cyclic or bicyclic ring system having three to ten carbon atoms and one to three rings, wherein each five-membered ring has one double bond, each six-membered ring has one or two double bonds, each seven- and eight-membered ring has one to three double bonds, and each nine-to ten-membered ring has one to four double bonds. Examples of cycloalkenyl groups include, but are not limited to, cyclohexenyl, octahydronaphthalenyl, norbornylenyl.

The term “cycloalkyl,” as used herein, refers to a saturated monocyclic, bicyclic, or tricyclic hydrocarbon ring system having three to twelve carbon atoms. Examples of cycloalkyl groups include, but are not limited to, cyclopropyl, cyclopentyl, bicyclo [3.1.1 I]heptyl, and adamantyl.

The term “(cycloalkyl)alkyl,” as used herein refers to a cycloalkyl group attached to the parent molecular moiety through an alkyl group.

The term “dialkylamino,” as used herein, refers to —NR^{c}R^{d}, wherein R^{c} and R^{d} are alkyl.

The term “dialkylaminoalkyl,” as used herein, refers to a dialkylamino group attached to the parent molecular moiety through an alkyl group.

The terms “halo” and “halogen,” as used herein, refer to F, Cl, Br, or I.

The term “haloalkoxy,” as used herein, refers to a haloalkyl group attached to the parent molecular moiety through an oxygen atom.

The term “haloalkoxyalkyl,” as used herein, refers to a haloalkoxy group attached to the parent molecular moiety through an alkyl group.

The term “haloalkyl,” as used herein, refers to an alkyl group substituted by at least one halogen atom.

The term “heteroaryl,” as used herein, refers to an aromatic five- or six-membered ring where at least one atom is selected from the group consisting of N, O, and S, and the remaining atoms are carbon. The five-membered rings have two double bonds, and the six-membered rings have three double bonds. The heteroaryl groups are connected to the parent molecular group through a substitutable carbon or nitrogen atom in the ring. The term “heteroaryl” also includes bicyclic systems where a heteroaryl ring is fused to a phenyl group, a monocyclic cycloalkenyl group, as defined herein, a monocyclic cycloalkyl group, as defined herein, a heterocycle group, as defined herein, or an additional heteroaryl group; and tricyclic systems where a bicyclic system is fused to a phenyl group, a monocyclic cycloalkenyl group, as defined herein, a monocyclic cycloalkyl group, as defined herein, a heterocycle group, as defined herein, or an additional heteroaryl group. Examples of heteroaryl groups include, but are not limited to, benzothienyl, benzoxadiazolyl, cinnolinyl, dibenzofuranyl, furanyl, imidazolyl, indazolyl, indolyl, isoxazolyl, isoquinolinyl, isothiazolyl, naphthyridinyl, oxadiazolyl, oxadiazolyl, oxazolyl, thiazolyl, thienopyridinyl, thienyl, triazolyl, thiadiazolyl, pyridinyl, pyridazinyl, pyrimidinyl, pyrazinyl, pyrazolyl, pyrrolyl, quinolinyl, and triazinyl. The heteroaryl groups of the present invention can be optionally substituted with one, two, three, four, or five substituents independently selected from the group consisting of alkoxy, alkoxyalkyl, alkoxycarbonyl, alkyl, alkylcarbonyl, alkylsulfanyl, amino, aminoalkyl, aminocarbonyl, cyano, cyanoalkyl, halo, haloalkoxy, haloalkyl, nitro, and oxo.

The term “heteroarylalkyl,” as used herein, refers to a heteroaryl group attached to the parent molecular moiety through an alkyl group.

The term “heterocycle,” as used herein, refers to cyclic, non-aromatic, five-, six-, or seven-membered rings containing at least one atom selected from the group consisting of oxygen, nitrogen, and sulfur. The five-membered rings have zero or one double bonds and the six- and seven-membered rings have zero, one, or two double bonds. The heterocycle groups of the invention are connected to the parent molecular group through a substitutable carbon or nitrogen atom in the ring. The term “heterocycle” also includes bicyclic systems where a heterocycle ring is fused to a phenyl group, a monocyclic cycloalkenyl group, as defined herein, a monocyclic cycloalkyl group, as defined herein, or an additional monocyclic heterocycle group; and tricyclic systems where a bicyclic system is fused to a phenyl group, a monocyclic cycloalkenyl group, as defined herein, a monocyclic cycloalkyl group, as defined herein, or an additional monocyclic heterocycle group. Examples of heterocycle groups include, but are not limited to, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, and thiomorpholinyl. The heterocycle groups of the present invention can be optionally substituted with one, two, three, four, or five substituents independently selected from the group consisting of alkoxy, alkoxyalkyl, alkoxycarbonyl, alkyl, alkylcarbonyl, alkylsulfanyl, amino, aminoalkyl, aminocarbonyl, cyano, cyanoalkyl, halo, haloalkoxy, haloalkyl, nitro, and oxo.

The term “(heterocycle)alkyl,” as used herein, refers to a heterocycle group attached to the parent molecular group through an alkyl group.

The term “hydroxy,” as used herein, refers to —OH.

The term “hydroxyalkyl,” as used herein, refers to an alkyl group substituted by at least one hydroxy group.

The term “nitro,” as used herein, refers to —NO₂.

The term “nitroalkyl,” as used herein, refers to an alkyl group substituted by at least one nitro group.

The compounds of the present invention can exist as therapeutically acceptable salts. The term “therapeutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate, lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate,trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

The present compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to parent compounds of formula (I) for example, by hydrolysis in blood.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, or mixtures thereof, which possess the ability to inhibit angiogenesis. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

In accordance with methods of treatment and pharmaceutical compositions of the invention, the compounds can be administered alone or in combination with other chemotherapeutic agents. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrastemal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The antiangiogenic effect of parenterally administered compounds can be prolonged by slowing their absorption. One way to slow the absorption of a particular compound is administering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefore.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Determination of Biological Activity

In Vitro Assay for Angiogenic Activity

The human microvascular endothelial (HMVEC) migration assay was run according to the procedure of S. S. Tolsma, 0. V. Volpert, D. J. Good, W. F. Frazier, P. J. Polyerini and N. Bouck, J. Cell Biol. 122, 497-511 (1993).

The HMVEC migration assay was carried out using Human Microvascular Endothelial Cells-Dermal (single donor) and Human Microvascular Endothelial Cells, (neonatal). The BCE or HMVEC cells were starved overnight in DME containing 0.01% bovine serum albumin (BSA). Cells were then harvested with trypsin and resuspended in DME with 0.01% BSA at a concentration of 1.5×10⁶ cells per mL. Cells were added to the bottom of a 48 well modified Boyden chamber (Nucleopore Corporation, Cabin John, Md.). The chamber was assembled and inverted, and cells were allowed to attach for 2 hours at 37° C. to polycarbonate chemotaxis membranes (5 μm pore size) that had been soaked in 0.01% gelatin overnight and dried. The chamber was then reinverted, and test substances (total volume of 50 μL), including activators, 15 ng/mL bFGF/VEGF, were added to the wells of the upper chamber. The apparatus was incubated for 4 hours at 37° C. Membranes were recovered, fixed and stained (Diff Quick, Fisher Scientific) and the number of cells that had migrated to the upper chamber per 3 high power fields counted. Background migration to DME+0.1 BSA was subtracted and the data reported as the number of cells migrated per 10 high power fields (400×) or, when results from multiple experiments were combined, as the percent inhibition of migration compared to a positive control.

Representative compounds described in Examples 1 to 171 inhibited human endothelial cell migration in the above assay by at least about 50% when tested at a concentration of 1 nM. Preferred compounds inhibited human endothelial cell migration by about 80 to about 95 percent when tested at a concentration of 1 nM.

Many diseases (characterized as “angiogenic diseases”) are driven by persistent unregulated angiogenesis. For example, ocular neovascularization has been implicated as the most common cause of blindness. In certain existing conditions such as arthritis, newly formed capillary blood vessels invade the joints and destroy cartilage. In diabetes, new capillaries formed in the retina invade the vitreous, bleed, and cause blindness. For example, ocular neovascularization has been implicated as the most common cause of blindness. In certain existing conditions such as arthritis, newly formed capillary blood vessels invade the joints and destroy cartilage. In diabetes, new capillaries formed in the retina invade the vitreous, bleed, and cause blindness. Growth and metastasis of solid tumors are also angiogenesis-dependent (Folkman, J., Cancer Res., 46: 467-473 (1986), Folkman, J., J. Natl. Cancer Inst., 82: 4-6 (1989)). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites, such as the liver, the lung, and the bones (Weidner, N., et. al., N. Engl. J. Med., 324(1): 1-8 (1991)).

The compounds of the invention, including not limited to those specified in the examples, possess antiangiogenic activity. As angiogenesis inhibitors, such compounds are useful in the treatment of both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). Such compounds may also be useful in treating solid tumors arising from hematopoietic malignancies such as leukemias (i.e., chloromas, plasmacytomas and the plaques and tumors of mycosis fungicides and cutaneous T-cell lymphoma/leukemia) as well as in the treatment of lymphomas (both Hodgkin's and non-Hodgkin's lymphomas). In addition, these compounds may be useful in the prevention of metastases from the tumors described above either when used alone or in combination with radiotherapy and/or other chemotherapeutic agents. The compounds of the invention can also be useful in the treatment of the aforementioned conditions by mechanisms other than the inhibition of angiogenesis.

Further uses include the treatment and prophylaxis of autoimmune diseases such as rheumatoid, immune and degenerative arthritis; various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemagiomas, and capillary proliferation within atherosclerotic plaques; Osler-Webber Syndrome; myocardial angiogenesis; plaque neovascularization; telangiectasia; hemophiliac joints; angiofibroma; and wound granulation. Other uses include the treatment of diseases characterized by excessive or abnormal stimulation of endothelial cells, including not limited to intestinal adhesions, Crohn's disease, atherosclerosis, scleroderma, and hypertrophic scars, i.e., keloids. Another use is as a birth control agent, by inhibiting ovulation and establishment of the placenta. The compounds of the invention are also useful in the treatment of diseases that have angiogenesis as a pathologic consequence such as cat scratch disease (Rochele minutesalia quintosa) and ulcers (Helicobacter pylori). The compounds of the invention are also useful to reduce bleeding by administration prior to surgery, especially for the treatment of resectable tumors.

Synthetic Methods

Abbreviations which have been used in the descriptions of the scheme and the examples that follow are: PPh₃ for triphenylphosphine; THF for tetrahydrofuran; DMSO for dimethylsulfoxide; and TFA for trifluoroacetic acid.

The compounds and processes of the present invention will be better understood in connection with the following synthetic schemes which illustrate the methods by which the compounds of the invention may be prepared. Starting materials can be obtained from commercial sources or prepared by well-established literature methods known to those of ordinary skill in the art. The groups R¹, R², R³, R⁴, R⁵, and R⁶ are as defined above unless otherwise noted below.

This invention is intended to encompass compounds having formula (I) when prepared by synthetic processes or by metabolic processes. Preparation of the compounds of the invention by metabolic processes include those occurring in the human or animal body (in vivo) or processes occurring in vitro.

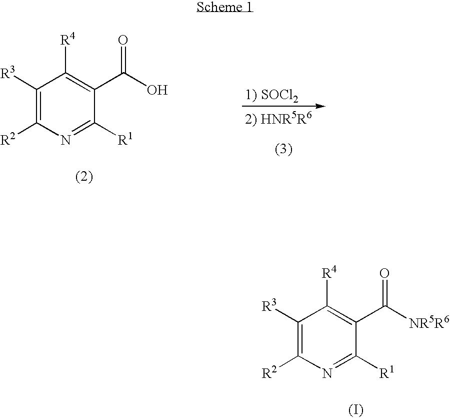

Scheme 1 shows the synthesis of compounds of formula (I). Compounds of formula (2) can be converted to the corresponding acid chloride by treatment with thionyl chloride. Examples of solvents used in this reaction include dichloromethane, chloroform, and carbon tetrachloride. The reaction is typically conducted at about −5° C. to about 15° C. for about 30 minutes to about 2 hours. The acid chloride can then be reacted with an appropriately substituted amine in the presence of a base such as triethylamine or diisopropylethylamine to provide compounds of formula (I). Examples of solvents used in this reaction include dichloromethane, chloroform, and carbon tetrachloride. The reaction is typically run at about 0° C. to about 40° C. for about 2 to about 6 hours.

Compounds of formula (2) can also be converted to compounds of formula (I) by treatment with compounds of formula (3) in the presence of a coupling reagent such as DCC, HOBT, and other coupling reagents known to those of ordinary skill in the art.

Compounds of formula (I) where one or more of R¹, R², R³, and R⁴ is halo can be coupled with an organoborane (in the presence of a base such as sodium carbonate or cesium fluoride), an organostannane, or an organozinc reagent in the presence of a palladium catalyst such as Pd(PPh₃)₄, PdCl₂(PPh₃)₂, or [1,1′-bis(diphenylphosphino)ferrocene]dichloropalladium optionally in the absence of CuI to provide compounds where one or more of R1, R², R³, and R⁴ is alkoxycarbonylalkyl, alkyl, aryl, arylalkyl, cyanoalkyl, cycloalkyl, (cycloalkyl)alkyl, or heteroaryl. Examples of solvents used in these reactions include dichloromethane, toluene, and THF. The reaction is typically conducted at about 25° C. to about 170° C. (depending on the conditions used) for about 8 to about 24 hours.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects.

Compounds of the invention were named by ACD/ChemSketch version 5.0 (developed by Advanced Chemistry Development, Inc., Toronto, ON, Canada) or were given names consistent with ACD nomenclature.

### EXAMPLE 1

### N,N-diethyl-6-methylnicotinamide

6-Methylnicotinic acid (8.25 g, 60 mmol) was suspended in dry dichloromethane (90 mL), cooled to 0° C., and treated with thionyl chloride (9 mL, 124 mmol). The mixture was stirred for one hour, and the excess reagent and solvent were removed in vacuo. The obtained acid chloride was then added dropwise to a solution of N,N-diethylamine (6.25 mL, 60 mmol) and triethylamine (45 mL) in dichloromethane (200 mL) at 0° C. The mixture was stirred for 4 hours and concentrated in vacuo. The resulting residue was dissolved in dichloromethane, and washed sequentially with saturated sodium bicarbonate, water, and brine. The combined extracts were dried over MgSO₄ and filtered. The filtrate was concentrated in vacuo and the residue was purified on a silica gel column eluting first with dichloromethane and then with a mixture of (99:1) dichloromethane/methanol. The resulting product was dissolved in diethyl ether, treated with 2 M HCl in diethyl ether (80 mL), and filtered. The filter cake was washed with diethyl ether, dried under vacuum, and recrystallized from methanol/ethyl acetate/hexane to provide the desired product (8.04 g) as the hydrochloride salt. MS m/e 193.0 (M+H)⁺; ¹H NMR (DMSO-d₆) δ 1.12 (d, 6H), 2.76 (s, 3H), 3.34 (dd, 4H), 7.88 (d, 1H), 8.37 (dd, 1H), 8.80 (d, 1H).

### EXAMPLE 2

### N,N-dimethyl-6-(1H-pyrazol-1-yl)nicotinamide

The desired product was prepared by substituting 6-(1H-pyrazol-1-yl)nicotinic acid for 6-methylnicotinic acid and N,N-dimethylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 217 (M+H)⁺; ¹H NMR (DMSO-d₆) δ 3.01 (d, 6H), 6.61-6.63 (m, 1H), 7.88 (d, 1H), 7.97 (d, 1H), 8.06 (dd, 1H), 8.54 (d, 1H), 8.66 (d, 1H).

### EXAMPLE 3

### N-ethyl-6-methylnicotinamide

The desired product was prepared by substituting ethylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 165 (M+H)⁺; ¹H NMR (DMSO-d₆) δ 0.84 (t, 3H), 2.49 (s, 3H), 2.96-3.07 (m, 2H), 7.67 (d, 1H), 8.54 (dd, 1H), 8.89 (d, 1H), 9.02 (br t, 1H).

### EXAMPLE 4

### N-ethyl-2-methylnicotinamide

The desired product was prepared by substituting 2-methylnicotinic acid for 6-methylnicotinic acid and ethylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 165 (M+H)⁺; ¹H NMR (DMSO-d₆) δ 1.12 (t, 3H), 2.50 (s, 3H), 3.21-3.30 (m, 2H), 7.23-7.28 (m, 1H), 7.67 (dd, 1H), 8.37 (br t, 1H), 8.48 (dd, 1H).

### EXAMPLE 5

### N-ethyl-5-methylnicotinamide

The desired product was prepared by substituting 5-methylnicotinic acid for 6-methylnicotinic acid and ethylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 165 (M+H)⁺; ¹H NMR (DMSO-d₆) δ 1.13 (t, 3H), 2.35 (s, 3H), 3.26-3.34 (m, 2H), 7.98-8.01 (m, 1H), 8.53 (d, 1H), 8.58 (brt, 1H), 8.80 (d, 1H).

### EXAMPLE 6

### N-butyl-N,6-dimethylnicotinamide

The desired product was prepared by substituting N-butyl-N-methylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 207 (M+H)⁺; ¹H NMR (DMSO-d₆) δ 0.76 (br t, 1H), 0.88-0.97 (m, 2H), 1.06-1.15 (m, 1H), 1.29-1.40 (m, 1H), 1.44-1.62 (m, 2H), 2.58 (s, 3H), 2.93 (d, 3H), 3.18 (br t, 1H), 3.45 (br t, 1H), 7.50-7.59 (m, 1H), 7.96 (dd, 1H), 8.61 (d, 1H).

### EXAMPLE 7

### N-isobutyl-N,6-dimethylnicotinamide

The desired product was prepared by substituting N-isobutyl-N-methylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 207 (M+H)⁺; ¹H NMR (DMSO-d₆) δ 0.70 (d, 2H), 0.88-0.96 (m, 4H), 1.82-2.08 (br m, 1H), 2.59 (s, 3H), 2.94 (d, 3H), 3.01-3.09 (m, 1H), 3.30 (d, 1H), 7.51-7.59 (m, 1H), 7.97 (dd, 1H), 8.62 (d, 1H).

### EXAMPLE 8

### N,6-dimethyl-N-pentylnicotinamide

The desired product was prepared by substituting N-methyl-N-pentylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 221 (M+H)⁺; ¹H NMR (DMSO-d₆) δ 0.79 (t, 1H), 0.87-0.93 (m, 1H), 1.01-1.09 (m, 1H), 1.11-1.19 (m, 1H), 1.25-1.39 (br m, 3H), 1.46-1.54 (m, 1H) 1.55-1.63 (m, 1H), 2.58 (s, 3H), 2.93 (d, 3H), 3.12-3.21 (m, 1H), 3.38-3.44 (m, 1H), 7.44-7.58 (m, 1H), 7.96 (dd, 1H), 8.62 (d, 1H).

### EXAMPLE 9

### N,6-dimethyl-N-(3-methylbutyl)nicotinamide

The desired product was prepared by substituting N-methyl-N-(3-methyl)butylamine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 207 (M+H)⁺; ¹H NMR (DMSO-d₆) δ 0.72 (d, 2H), 0.94 (d, 4H), 1.38-1.51 (br m, 2H), 1.56-1.67 (br m, 1H), 2.59 (s, 3H), 2.93 (d, 3H), 3.18 (br t, 1H), 3.46 (br t, 1H), 7.50-7.59 (m, 1H), 7.98 (dd, 1H), 8.63 (d, 1H).

### EXAMPLE 10

### N-(cyanomethyl)-N,6-dimethylnicotinamide

The desired product was prepared by substituting (methylamino)acetonitrile for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 190 (M+H)⁺; ¹H NMR (DMSO-d₆) δ 2.65 (s, 3H), 3.05 (s, 3H), 4.55 (s, 2H), 7.42 (d, 1H), 7.88 (d, 1H), 8.61 (s, 1H).

### EXAMPLE 11

### N-cyclohexyl-N,6-dimethylnicotinamide

The desired product was prepared by substituting N-cyclohexyl-N-methylamine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 233 (M+H)⁺; ¹H NMR (DMSO-d₆) δ 0.98-1.18 (br m, 2H), 1.30-1.41 (br m, 1H), 1.45-1.86 (br m, 7H), 2.61 (s, 3H), 2.83 (d, 3H), 3.27 (br t, 0.5H), 4.28 (br t, 0.5H), 7.62 (br t, 1H), 8.05 (dd, 1H), 8.67 (d, 1H).

### EXAMPLE 12

### N-butyl-N-isopropyl-6-methylnicotinamide

The desired product was prepared by substituting N-butyl-N-isopropylamine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 235 (M+H)⁺; ¹H NMR (DMSO-d₆) δ 0.61-1.61 (br m, 13H), 2.59 (s, 3H), 3.09 (br s, 0.5H), 3.26 (br s, 1.5H), 3.74 (br s, 0.75H), 4.39 (br s, 0.25H), 7.57 (d, 1H), 7.97 (d, 1H), 8.61 (s, 1H).

### EXAMPLE 13

### 6-methyl-N,N-dipropylnicotinamide

The desired product was prepared by substituting N,N-dipropylamine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 221 (M+H)⁺; ¹H NMR (DMSO-d₆) δ 0.71-0.63 (br t, 3H), 0.84-0.95 (br t, 3H), 1.43-1.66 (br d, 4H), 2.53 (s, 3H), 3.08-3.18 (br t, 2H), 3.32-3.42 (br t, 2H), 7.40 (d, 1H), 7.77 (d, 1H), 8.46 (d, 1H).

### EXAMPLE 14

### N-isopropyl-6-methyl-N-propylnicotinamide

The desired product was prepared by substituting N-isopropyl-N-propylamine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 221 (M+H)⁺; ¹H NMR (DMSO-d₆) δ 0.63 (br s,1H), 0.85-0.97 (br m, 2H), 1.05-1.69 (br m, 8H), 2.60 (s, 3H), 2.99-3.29 (br m, 2H), 3.74 (br s,0.75H), 4.40 (br s,0.25H), 7.60 (d, 1H), 8.00 (d, 1H), 8.63 (s, 1H).

### EXAMPLE 15

### N-butyl-6-methyl-N-propylnicotinamide

The desired product was prepared by substituting N-butyl-N-propylamine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 235 (M+H)⁺; ¹H NMR (DMSO-d₆) δ 0.63-0.79 (br m, 3H), 0.85-0.99 (br m, 3H), 1.03-1.14 (br m, 1H), 1.27-1.39 (br m, 1H), 1.53 (br d. 4H), 2.60 (s, 3H), 3.05-3.19 (br m, 2H), 3.33-3.47 (br m, 2H), 7.58 (d, 1H), 7.99 (dd, 1H), 8.62 (d, 1H).

### EXAMPLE 16

### N-isopropyl-N-(2-methoxyethyl)-6-methylnicotinamide

The desired product was prepared by substituting N-isopropyl-N-(2-methoxyethyl)amine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 237 (M+H)⁺; ¹H NMR (DMSO-d₆) δ 0.97-1.36 (br m, 6H), 2.56 (s, 3H), 3.05-3.59 (br m, 7H), 3.75 (br s,1H), 7.49 (d, 1H), 7.87 (br s,1H), 8.55 (s, 1H).

### EXAMPLE 17

### N-butyl-N-(cyanomethyl)-6-methylnicotinamide

The desired product was prepared by substituting (butylamino)acetonitrile for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 232 (M+H)⁺; ¹H NMR (DMSO-d₆) δ 0.78 (br m, 3H), 1.14 (br s, 2H), 1.48-1.61 (br m, 2H), 2.55 (s, 3H), 3.23-3.40 (m, 2H), 4.51 (s, 2H), 7.45 (d, 1H), 7.86 (dd, 1H), 8.57 (d, 1H).

### EXAMPLE 18

### N,6-dimethyl-N-(tetrahydro-2-furanylmethyl)nicotinamide

The desired product was prepared by substituting N-methyl-N-(tetrahydro-2-furanylmethyl)amine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 235 (M+H)⁺; ¹H NMR (DMSO-d₆) δ 1.44-2.02 (br m, 4H), 2.57 (s, 3H), 2.99 (d, 3H), 3.17-3.31 (br m, 1H), 3.34-3.44 (m, 0.5H), 3.45-3.55 (m, 0.5H), 3.56-3.71 (br m, 2H), 3.74-3.81 (m, 0.5H), 3.99-4.07 (br m, 0.5H), 7.52 (d, 1H), 7.94 (dd, 1H), 8.59 (br s, 1H).

### EXAMPLE 19

### 2-chloro-N-ethyl-N-isopropyl-6-methylnicotinamide

The desired product was prepared by substituting 2-chloro-6-methylnicotinic acid for 6-methylnicotinic acid and N-ethyl-N-isopropylamine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 240.9 (M+H)⁺; ¹H NMR (DMSO-d₆) δ 0.94 (t, 1H), 1.05 (d, 2H), 1.15-1.21 (m, 5H), 1.24 (br d, 2H), 2.48 (s, 3H), 3.39-3.47 (m, 1H), 3.48-3.55 (m, 0.7H), 4.46-4.53 (m, 0.3H), 7.35 (d, 1H), 7.73 (d, 0.7H), 7.77 (d, 0.3H).

### EXAMPLE 20

### N-[2-(dimethylamino)ethyl]-N,6-dimethylnicotinamide

The desired product was prepared by substituting N-[2-(dimethylamino)ethyl]-N-methylamine for N,N-diethylamine in Example 1 and scaling the reaction to a 1 mmol scale. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 222 (M+H)⁺; ¹H NMR (DMSO-d₆) δ 2.55 (s, 3H), 2.79-3.00 (br m, 9H), 3.30-3.42 (br m, 2H), 3.73-3.86 (br m, 2H), 7.43 (d, 1H), 7.89 (d, 1H), 8.60 (s, 1H).

### EXAMPLE 21

### 2-chloro-N,N,6-trimethylnicotinamide

The desired product was prepared by substituting 2-chloro-6-methylnicotinic acid for 6-methylnicotinic acid and N,N-dimethylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as trifluoroacetate salt. MS m/e 164.9 (M+H)⁺; ¹H NMR (DMSO-d₆) δ 2.71 (s, 3H), 2.98 (d, 6H), 7.82 (d, 1H), 8.33 (dd, 1H), 8.81 (d, 1H).

### EXAMPLE 22

### N-[2-(dimethylamino)ethyl]-N-ethyl-6-methylnicotinamide

The desired product was prepared by substituting N-[2-(dimethylamino)ethyl]-N-ethylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 236.2 (M+H)⁺; ¹H NMR (DMSO-d₆) δ 1.07 (t, 3H), 2.54 (s, 3H), 2.88 (br s, 6H), 3.19-3.40 (br m, 4H), 3.68-3.80 (br m, 2H), 7.41 (d, 1H), 7.82 (dd, 1H), 8.55 (d, 1H).

### EXAMPLE 23

### 2-chloro-N-cyclohexyl-N-ethyl-6-methylnicotinamide

The desired product was prepared by substituting 2-chloro-6-methylnicotinic acid for 6-methylnicotinic acid and N-cyclohexyl-N-ethylamine for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 280.9 (M+H)⁺; ¹H NMR (DMSO-d₆) δ 0.86-0.95 (m, 1.75H), 0.98-1.06 (br m, 1H), 1.11-1.19 (m, 2.25H), 1.26-1.38 (br m, 1H), 1.43-1.83 (br m, 7H), 2.49 (d, 3H), 2.99-3.07 (m, 0.75H), 3.27-3.51 (m, 2H), 4.12-4.19 (m, 0.25H), 7.34 (d, 1H), 7.73 (dd, 0.65H), 7.76 (dd, 0.35H).

### EXAMPLE 24

### N,N-diethyl-2-methyl-6-(trifluoromethyl)nicotinamide

The desired product was prepared by substituting 2-methyl-6-trifluoromethylnicotinic acid for 6-methylnicotinic acid in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt.

### EXAMPLE 25

### 6-(2,2,2-trifluoroethoxy)nicotinamide

The desired product was prepared by substituting 6-(2,2,2-trifluoroethoxy)nicotinic acid for 6-methylnicotinic acid and ammonia for N,N-diethylamine in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 221.1 (M+H)⁺; ¹H NMR (DMSO-d₆) δ 5.06 (q, 2H), 7.06 (d, 1H), 7.51 (br s, 1H), 8.06 (br s, 1H), 8.32 (dd, 1H), 8.70 (d, 1H).

### EXAMPLE 26

### N,N-diethylnicotinamide

The desired product was prepared by substituting nicotinic acid for 6-methylnicotinic acid in Example 1 and scaling the reaction to a 1 mmol scale. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 179 (M+H)⁺; ¹H NMR (DMSO-d₆) δ 1.09 (d, 6H), 3.32 (dd, 4H), 7.40 (br s, 1H), 7.97-8.04 (m, 1H), 8.43-8.48 (m, 1H), 8.91 (dd, 1H).

### EXAMPLE 27

### N,N-diethyl-5-(2-methylphenyl)nicotinamide

A solution of 5-bromo-N,N-diethylnicotinamide (1 mmol), (prepared by substituting 5-bromonicotinic acid for 6-methylnicotinc acid in Example 1 and scaling the reaction to a 1 mmol scale), 2-methylphenylboronic acid (2.0 mmol), and tetrakis(triphenylphosphine)palladium (0) (0.05 mmol) in 1,2-dimethoxyethane (1.5 mL) and ethanol (0.25 mL), was treated with a solution of 2 M sodium carbonate (0.5 mL), heated to 87° C. overnight, and concentrated in vacuo. The residue was dissolved in diethyl ether and washed with water three times. The combined extracts were dried (Na₂SO₄), filtered, and concentrated in vacuo. The residue was purified by HPLC on a C-18 column and a solvent system increasing in gradient over 50 minutes from 5% to 100% acetonitrile/water containing 0.01% TFA and lyophilized to provide the desired product as the trifluoroacetate salt. MS m/e 269.2 (M+H)⁺; ¹H NMR (DMSO-d₆) δ 1.13 (br d, 6H), 2.25 (s, 3H), 3.25 (br s, 2H), 3.48 (br s, 2H), 7.26-7.38 (m, 4H), 7.78 (t, 1H), 8.59 (dd, 2H).

### EXAMPLE 28

### methyl 4-{5-[(diethylamino)carbonyl]-3-pyridinyl} benzoate

The desired product was prepared by substituting 4-(methoxycarbonyl)phenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 313.2 (M+H)⁺; ¹H NMR (DMSO-d₆) δ 1.14 (br d, 6H), 3.24 (br s, 2H), 3.49 (br s, 2H), 3.89 (s, 3H), 7.94-7.99 (m, 2H), 8.05-8.11 (m, 2H), 8.16 (t, 1H), 8.62 (d, 1H), 9.03 (d, 1H).

### EXAMPLE 29

### 5-(3-aminophenyl)-N,N-diethylnicotinamide

The desired product was prepared by substituting 3-aminophenylboronic acid for 2-methylphenylboronic acid in Example 27. After workup the crude compound was purified by HPLC on a C-18 column and a solvent system increasing in gradient over 50 minutes from 5% to 100% acetonitrile/water containing 0.01% TFA to provide the desired product as the trifluoroacetate salt. MS m/e 270.2 (M+H)⁺; ¹H NMR (DMSO-d₆) δ 1.13 (br d, 6H), 3.24 (br s, 2H), 3.47 (br s, 2H), 5.32 (br s, 2H), 6.63-6.67 (m, 1H), 6.86-6.90 (m, 1H), 6.92 (t, 1H), 7.89 (t, 1H), 8.51 (d, 1H), 8.83 (d, 1H).

### EXAMPLE 30

### N,N-diethyl-5-(2-methoxyphenyl)nicotinamide

The desired product was prepared by substituting 2-methoxyphenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 285.2 (M+H)⁺; ¹H NMR (DMSO-d₆) δ 1.14 (br d, 6H), 3.26 (br s, 2H), 3.48 (br s, 2H), 3.80 (s, 3H), 7.06-7.11 (m, 1H), 7.17 (dd, 1H), 7.39-7.46 (m, 2H), 7.88 (t, 1H), 8.52 (d, 1H), 8.73 (d, 1H).

### EXAMPLE 31

### N,N-diethyl-5-(4-methoxyphenyl)nicotinamide

The desired product was prepared by substituting 4-methoxyphenylboronic acid for 2-methylphenylboronic acid in Example 27. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 285.2 (M+H)⁺; ¹H NMR (DMSO-d₆) δ 1.13 (br d, 6H), 3.24 (br s, 2H), 3.47 (br s, 2H), 3.82 (s, 3H), 7.05-7.09 (m, 2H), 7.72-7.76 (m, 2H), 8.02 (t, 1H), 8.50 (d, 1H), 8.92 (d, 1H).

### EXAMPLE 32

### N,N-diethyl-5-(3-fluorophenyl)nicotinamide

The desired product was prepared by substituting 3-fluorophenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 273.2 (M+H)⁺; ¹H NMR (DMSO-d₆) δ 1.00-1.27 (br m, 6H), 3.23 (br d, 2H), 3.49 (br d, 2H), 7.25-7.31 (m, 1H), 7.53-7.59 (m, 1H), 7.64-7.73 (m, 2H), 8.13 (t, 1H), 8.58 (d, 1H), 9.00 (d, 1H).

### EXAMPLE 33

### N,N-diethyl-5-(4-fluorophenyl)nicotinamide

The desired product was prepared by substituting 4-fluorophenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 273.2 (M+H)⁺; ¹H NMR (DMSO-d₆) δ 1.13 (br d, 6H), 3.23 (br d, 2H), 3.48 (br d, 2H), 7.32-7.39 (m, 2H), 7.82-7.89 (m, 2H), 8.07 (t, 1H), 8.55 (d, 1H), 8.95 (d, 1H).

### EXAMPLE 34

### 5-(3-chlorophenyl)-N,N-diethylnicotinamide

The desired product was prepared by substituting 3-chlorophenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 289.2 (M+H)⁺; ¹H NMR (DMSO-d₆) δ 1.13 (br d, 6H), 3.23 (br d, 2H), 3.49 (br d, 2H), 7.49-7.58 (m, 2H), 7.75-7.79 (m, 1H), 8.13 (t, 1H), 8.58 (d, 1H), 8.99 (d, 1H).

### EXAMPLE 35

### 5-(2-bromophenyl)-N,N-diethylnicotinamide

The desired product was prepared by substituting 2-bromophenylboronic acid for 2-methylphenylboronic acid in Example 27. After workup the crude compound was purified by HPLC on a C-18 column using a solvent system increasing in gradient over 50 minutes from 5% to 100% acetonitrile/water containing 0.01% TFA to provide the trifluoroacetate salt. MS m/e 333.1 (M+H)⁺; ¹H NMR (DMSO-d₆) δ 1.13 (br d, 6H), 3.27 (br d, 2H), 3.47 (br d, 2H), 7.38-7.43 (m, 1H), 7.48-7.53 (m, 2H), 7.79 (dd, 1H), 7.83 (t, 1H), 8.61 (d, 1H), 8.65 (d, 1H).

### EXAMPLE 36

### 5-(3-bromophenyl)-N,N-diethylnicotinamide

The desired product was prepared by substituting 3-bromophenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 333.1 (M+H)⁺; ¹H NMR (DMSO-d₆) δ 1.13 (br d, 6H), 3.23 (br d, 2H), 3.49 (br d, 2H), 7.47 (t, 1H), 7.62-7.67 (m, 11H), 7.79-7.83 (m, 1H), 8.02 (t, 11H), 8.58 (d, 1H), 8.98 (d, 1H).

### EXAMPLE 37

### 5-(3-cyanophenyl)-N,N-diethylnicotinamide

The desired product was prepared by substituting 3-cyanophenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 280.2 (M+H)⁺; ¹H NMR (DMSO-d₆) δ 1.13 (br d, 6H), 3.24 (br d, 2H), 3.49 (br d, 2H), 7.72 (t, 1H), 7.89-7.93 (m, 11H), 8.14-8.18 (m, 1H), 8.20 (t, 11H), 8.34 (t, 1H), 8.61 (d, 1H), 9.04 (d, 11H).

### EXAMPLE 38

### 5-(4-acetylphenyl)-N,N-diethylnicotinamide

The desired product was prepared by substituting 4-acetylphenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 297.2 (M+H)⁺; ¹H NMR (DMSO-d₆) δ 1.15 (br d, 6H), 2.63 (s, 3H), 3.24 (br d, 2H), 3.49 (br d, 2H), 7.94-7.99 (m, 2H), 8.05-8.10 (m, 2H), 8.17 (t, 1H), 8.61 (d, 1H), 9.04 (d, 1H).

### EXAMPLE 39

### 5-(2,5-dimethylphenyl)-N,N-diethylnicotinamide

The desired product was prepared by substituting 2,5-dimethylphenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 283.2 (M+H)⁺; ¹H NMR (DMSO-d₆) δ 1.13 (br d, 6H), 2.20 (s, 3H), 2.32 (s, 3H), 3.25 (br s, 2H), 3.47 (br s, 2H), 7.10 (s, 1H), 7.15 (dd, 1H), 7.23 (d, 1H), 7.76 (t, 1H), 8.56 (d, 1H), 8.61 (d, 1H).

### EXAMPLE 40

### 5-(3,4-dimethylphenyl)-N,N-diethylnicotinamide

The desired product was prepared by substituting 3,4-dimethylphenylboronic acid for 2-methylphenylboronic acid in Example 27 After workup the crude compound was purified by HPLC on a C-18 column and a solvent system increasing in gradient from 5% to 100% acetonitrile/water containing 0.01% TFA over 50 minutes to provide the desired product as the trifluoroacetate salt. MS m/e 283.2 (M+H)⁺; ¹H NMR (DMSO-d₆) δ 1.13 (br d, 6H), 2.28 (s, 3H), 2.31 (s, 3H), 3.23 (br d, 2H), 3.48 (br d, 2H), 7.27 (d, 1H), 7.51 (dd, 1H), 7.59 (s, 1H), 8.08 (t, 1H), 8.55 (d, 1H), 8.95 (d, 1H).

### EXAMPLE 41

### 5-(3,5-dimethylphenyl)-N,N-diethylnicotinamide

The desired product was prepared by substituting 3,5-dimethylphenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 283.2 (M+H)⁺; ¹H NMR (DMSO-d₆) δ 1.13 (br d, 6H), 2.35 (s, 6H), 3.23 (br d, 2H), 3.48 (br d, 2H), 7.09 (s, 1H), 7.40 (s, 2H), 8.07 (t, 1H), 8.56 (d, 1H), 8.94 (d, 1H).

### EXAMPLE 42

### 5-(3-ethoxyphenyl)-N,N-diethylnicotinamide

The desired product was prepared by substituting 3-ethoxyphenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 299.2 (M+H)⁺; ¹H NMR (DMSO-d₆) δ 1.14 (br d, 6H), 1.86 (t, 3H), 3.23 (br d, 2H), 3.49 (br d, 2H), 4.13 (q, 2H), 6.99-7.02 (m, 1H), 7.30-7.35 (m, 2H), 7.42 (t, 1H), 8.12 (t, 1H), 8.57 (d, 1H), 8.98 (d, 1H).

### EXAMPLE 43

### 5-(2,4-dimethoxyphenyl)-N,N-diethylnicotinamide

The desired product was prepared by substituting 2,4-dimethoxyphenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 315.2 (M+H)⁺; ¹H NMR (DMSO-d₆) δ 1.01-1.22 (br m, 6H), 3.14-3.30 (br m, 2H), 3.39-3.53 (br m, 2H), 3.68 (s, 3H), 3.83 (s, 3H), 6.77 (dd, 1H), 6.72 (d, 1H), 7.36 (d, 1H), 7.91 (t, 1H), 8.51 (d, 1H), 8.73 (d, 1H).

### EXAMPLE 44

### 5-(2,5-dimethoxyphenyl)-N,N-diethylnicotinamide

The desired product was prepared by substituting 2,5-dimethoxyphenylboronic acid for 2-methylphenylboronic acid in Example 27. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 315.2 (M+H)⁺; ¹H NMR (DMSO-d₆) δ 1.14 (br d, 6H), 3.26 (br s, 2H), 3.47 (br s, 2H), 3.74 (s, 3H), 3.77 (s, 3H), 6.97-7.03 (m, 2H), 7.08-7.14 (m, 1H), 7.95 (t, 1H), 8.55 (d, 1H), 8.77 (d, 1H).

### EXAMPLE 45

### 5-(3,4-dimethoxyphenyl)-N,N-diethylnicotinamide

The desired product was prepared by substituting 3,4-dimethoxyphenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 315.2 (M+H)⁺; ¹H NMR (DMSO-d₆) δ 1.44 (br d, 6H), 3.24 (br d, 2H), 3.49 (br d, 2H), 3.81 (s, 3H), 3.87 (s, 3H), 7.08 (d, 1H), 7.32-7.38 (m, 2H), 8.13 (t, 1H), 8.53 (d, 1H), 8.99 (d, 1H).

### EXAMPLE 46

### 5-[3-(acetylamino)phenyl]-N,N-diethylnicotinamide

The desired product was prepared by substituting 3-(acetylamino)phenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 312.2 (M+H)⁺; ¹H NMR (DMSO-d₆) δ 1.14 (br d, 6H), 2.07 (s, 3H), 3.24 (br s, 2H), 3.49 (br s, 2H), 7.42-7.47 (m, 2H), 7.61-7.66 (m, 1H), 7.97 (s, 1H), 8.00 (t, 1H), 8.60 (d, 1H), 8.91 (d, 11H), 10.06 (s, 1H).

### EXAMPLE 47

### N,N-diethyl-5-(3,4,5-trimethoxyphenyl)nicotinamide

The desired product was prepared by substituting 3,4,5-trimethoxyphenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 345.2 (M+H)⁺; ¹H NMR (DMSO-d₆) δ 1.02-1.26 (br m, 6H), 3.23 (br d, 2H), 3.49 (br d, 2H), 3.72 (s, 3H), 3.89 (s, 6H), 7.06 (s, 2H), 8.17 (t, 1H), 8.56 (d, 11H), 9.02 (d, 11H).

### EXAMPLE 48

### N,N-diethyl-3,4′-bipyridine-5-carboxamide

The desired product was prepared by substituting 4-pyridinylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 256.1 (M+H)⁺; ¹H NMR (DMSO-d₆) δ 1.01-1.27 (br m, 6H), 3.24 (br d, 2H), 3.50 (br d, 2H), 8.17 (dd, 2H), 8.34 (t, 1H), 8.72 (d, 1H), 8.86 (dd, 2H), 9.18 (d, 1H).

### EXAMPLE 49

### N,N-diethyl-5-(3-furyl)nicotinamide

The desired product was prepared by substituting 3-furylboronic acid for 2-methylphenylboronic acid in Example 27. After workup the crude compound was purified by HPLC on a C-18 column and a solvent system increasing in gradient from 5% to 100% acetonitrile/water containing 0.01% TFA over 50 minutes to provide the desired product as the trifluoroacetate salt. MS m/e 245.1 (M+H)⁺; ¹H NMR (DMSO-d₆) δ 1.13 (br d, 6H), 3.22 (br d, 2H), 3.48 (br d, 2H), 7.15 (dd, 1H), 7.81 (t, 1H), 8.08 (t, 1H), 8.42 (t, 1H), 8.46 (d, 1H), 8.96 (d, 1H).

### EXAMPLE 50

### N-isopropyl-N,6-dimethylnicotinamide

The desired product was prepared by substituting N-isopropyl-N-methylamine for N,N-diethylamine in Example 1. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 193.0 (M+H)⁺; ¹H NMR (DMSO-d₆) δ 1.07-1.22 (br m, 6H), 2.78 (s, 4.5H), 2.87 (s, 1.5H), 3.67-3.81 (br m, 0.5H), 4.60-4.78 (br m, 0.5H), 7.91 (d, 1H), 8.41 (dd, 1H), 8.82 (d, 11H).

### EXAMPLE 51

### N,N-dibutyl-6-methylnicotinamide

The desired product was prepared by substituting N,N-dibutylamine for N,N-diethylamine in Example 1. After workup the crude compound was purified by HPLC on a C-18 column and a solvent system increasing in gradient over 50 minutes from 5% to 100% acetonitrile/water containing 0.01% TFA to provide the desired product as the trifluoroacetate salt. MS m/e 249 (M+H)⁺; ¹H NMR (DMSO-d₆) δ 0.66-1.01 (m, 6H), 1.01-1.17 (br m, 2H), 1.27-1.66 (br m, 6H), 2.77 (s, 3H), 3.15 (br t, 2H), 3.42 (br t, 2H), 7.89 (d, 1H), 8.36 (dd, 1H), 8.80 (d, 1H).

### EXAMPLE 52

### 6-(4-aminophenyl)-N,N-diethylnicotinamide

The desired product was prepared by substituting 6-bromo-N,N-diethylnicotinamide for 5-bromo-N,N-diethylnicotinamide and 4-aminophenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 270.1 (M+H)⁺; ¹H NMR (DMSO-d₆) δ 1.13 (br s, 6H), 3.38 (br s, 2H), 3.48 (br s, 2H), 6.77 (d, 2H), 7.33-7.41 (m, 1H), 7.81 (dd, 1H), 7.84-7.91 (m, 4H), 8.55 (dd, 1H).

### EXAMPLE 53

### 6-(3-acetylphenyl)-N,N-diethylnicotinamide

The desired product was prepared by substituting 6-bromo-N,N-diethylnicotinamide for 5-bromo-N,N-diethylnicotinamide and 3-acetylphenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 297.2 (M+H)⁺; ¹H NMR (DMSO-d₆) δ 1.14 (br d, 6H), 2.67 (s, 3H), 3.26 (br s, 2H), 3.48 (br s, 2H), 7.68 (t, 1H), 7.93 (dd, 1H), 8.03-8.07 (m, 1H), 8.14 (dd, 1H), 8.35-8.39 (m, 1H), 8.67 (t, 1H), 8.70 (dd, 1H).

### EXAMPLE 54

### 6-[3-(acetylamino)phenyl]-N,N-diethylnicotinamide

The desired product was prepared by substituting 6-bromo-N,N-diethylnicotinamide for 5-bromo-N,N-diethylnicotinamide and 3-acetamidophenylboronic acid for 2-methylphenylboronic acid in Example 27. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 312.1 (M+H)⁺; ¹H NMR (DMSO-d₆) δ 1.14 (br d, 6H), 2.07 (s, 3H), 3.26 (br s, 2H), 3.48 (br s, 2H), 7.43 (t, 1H), 7.71 (dd, 1H), 7.73-7.77 (m, 1H), 7.89 (dd, 1H), 7.94 (dd, 1H), 8.36 (t, 1H), 8.66 (dd, 1H), 10.07 (s, 1H).

### EXAMPLE 55

### 6-(3,5-dichlorophenyl)-N,N-diethylnicotinamide

The desired product was prepared by substituting 6-bromo-N,N-diethylnicotinamide for 5-bromo-N,N-diethylnicotinamide and 3,5-dichlorophenylboronic acid for 2-methylphenylboronic acid in Example 27 After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 323.1 (M+H)ₜ; ¹H NMR (DMSO-d₆) δ 1.14 (br d, 6H), 3.23 (br s, 2H), 3.47 (br s, 2H), 7.71 (t, 1H), 7.93 (dd, 1H), 8.16-8.20 (m, 3H), 8.68 (dd, 1H).

### EXAMPLE 56

### N,N-diethyl-6-(2-thienyl)nicotinamide

The desired product was prepared by substituting 6-bromo-N,N-diethylnicotinamide for 5-bromo-N,N-diethylnicotinamide and 2-thienylboronic acid for 2-methylphenylboronic acid in Example 27. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 261.1 (M+H)⁺; ¹H NMR (DMSO-d₆) δ 1.13 (br s, 6H), 3.25 (br s, 2H), 3.45 (br s, 2H), 7.19 (q, 1H), 7.69 (dd, 1H), 7.83 (dd, 1H), 7.86 (dd, 1H), 7.97 (dd, 1H), 8.52 (dd, 1H).

### EXAMPLE 57

### 6-bromo-N,N-diethylnicotinamide

The desired product was prepared by substituting 6-bromonicotinic acid for 6-methylnicotinic acid in Example 1. After workup the crude compound was purified by HPLC on a C-18 column using a solvent system increasing in gradient over 50 minutes from 5% to 100% acetonitrile/water containing 0.01% TFA to provide the desired product as the trifluoroacetate salt. MS m/e 258.7 (M+H)⁺; ¹H NMR (DMSO-d₆) δ 0.92-1.25 (br m, 6H), 3.16 (br d, 2H), 3.43 (br d, 1H), 8.11 (t, 1H), 8.56 (d, 1H), 8.77 (d, 1H).

### EXAMPLE 58

### 6-sec-butyl-N,N-diethylnicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M 2-butylzinc bromide in THF (0.291 mmol). The mixture was heated in a single mode microwave synthesizer at 160° C. under nitrogen for 10 minutes and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient over 50 minutes from 5% to 100% acetonitrile/water containing 0.01% TFA to provide the desired product as the trifluoroacetate salt. MS m/e 235.2 (M+H)⁺; ¹H NMR (DMSO-d₆) δ 0.78 (t, 3H), 1.12 (br d, 6H), 1.24 (d, 3H), 1.55-1.65 (m, 1H), 1.66-1.77 (m, 1H), 2.80-2.91 (m, 1H), 3.21 (br s, 2H), 3.44 (br s, 2H), 7.41 (d, 1H), 7.83 (dd, 1H), 8.55 (d, 1H).

### EXAMPLE 59

### N,N-diethyl-6-(1-ethylpropyl)nicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M 3-pentylzinc bromide in THF (0.291 mmol). The mixture was heated in a single mode microwave synthesizer at 160° C. under nitrogen for 10 minutes and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient over 50 minutes from 5% to 100% acetonitrile/water containing 0.01% TFA to provide the desired product as the trifluoroacetate salt. MS m/e 249.2 (M+H)⁺; ¹H NMR (DMSO-d₆) 0.72 (t, 6H), 1.11 (br d, 6H), 1.62-1.73 (m, 4H), 2.59-2.68 (m, 1H), 3.21 (br s, 2H), 3.44 (br s, 2H), 7.38 (d, 11H), 7.83 (dd, 11H), 8.57 (d, 11H).

### EXAMPLE 60

### N,N-diethyl-6-hexylnicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M 1-hexylzinc bromide in T14F (0.291 mmol). The mixture was heated in a single mode microwave synthesizer at 160° C. under nitrogen for 10 minutes and concentrated in vacuo. The residue was dissolved in 1:1 DMSO/methanol and filtered through a membrane filter. The filtrate was concentrated in vacuo and the crude product was purified by 14PLC on a C-18 column using a solvent system increasing in gradient from 5% to 100% acetonitrile/water containing 0.01% TFA over 50 minutes to provide the desired product as the trifluoroacetate salt. MS m/e 263.2 (M+H)⁺; ¹H NMR (DMSO-d₆) δ 0.80-0.90 (m, 3H), 1.11 (br d, 6H), 1.23-1.36 (m, 6H), 1.63-1.73 (m, 2H), 2.80 (t, 2H), 3.20 (br s, 2H), 3.44 (br s, 2H), 7.46 (d, 1H), 7.86 (dd, 1H), 8.56 (d, 1H).

### EXAMPLE 61

### N,N-diethyl-6-(2-ethylbutyl)nicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M (2-ethyl)butylzinc bromide in THF (0.291 mmol). The mixture was heated in a single mode microwave synthesizer at 160° C. under nitrogen for 10 minutes, and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 263.2 (M+H)⁺; ¹H NMR (DMSO-d₆)δ 0.84 (t, 6H), 1.11 (br d, 6H), 1.22-1.33 (in, 4H), 1.71-1.81 (m, 1H), 2.74 (d, 2H), 3.20 (br s, 2H), 3.44 (br s, 2H), 7.43 (d, 1H), 7.84 (dd, 1H), 8.56 (d, 1H).

### EXAMPLE 62

### N,N-diethyl-6-(1-methylpentyl)nicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M 2-hexylzinc bromide in THF (0.291 mmol). The mixture was heated in a single mode microwave synthesizer at 160° C. under nitrogen for 10 minutes, and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 263.2 (M+H)⁺; ¹NMR (DMSO-d₆) δ 0.72 (t, 3H), 0.82(t, 3H), 1.12 (br d, 6H), 1.24 (d, 4H), 1.51-1.75 (m, 2H), 2.87-2.98 (m, 1H), 3.21 (br s, 2H), 3.44 (br s, 2H), 7.36-7.44 (m, 1H), 7.82 (dd, 1H), 8.52-8.56 (m, 11H).

### EXAMPLE 63

### N,N-diethyl-6-(1-ethylbutyl)nicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M 3-hexylzinc bromide in THF (0.291 mmol). The mixture was heated in a single mode microwave synthesizer at 160° C. under nitrogen for 10 minutes, and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 263.2 (M+H)⁺; ¹H NMR (DMSO-d₆) δ 0.72 (t, 3H), 0.82 (t, 3H), 0.99-1.20 (br m, 8H), 1.24 (d, 1H), 1.51-1.74 (m, 3H), 2.68-2.78 (m, 1H), 3.21 (br s, 2H), 3.45 (br s, 2H), 7.33-7.44 (m, 1H), 7.81 (dd, 1H), 8.52-8.59 (m, 1H).

### EXAMPLE 64

### 6-(cyclohexylmethyl)-N,N-diethylnicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M cyclohexylmethylzinc bromide in THF (0.291 mmol). The mixture was heated in a single mode microwave synthesizer at 160° C. under nitrogen for 10 minutes, and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 275.2 (M+H)⁺; ¹H NMR (DMSO-d₆) δ 0.88-1.25 (m, 1H), 1.51-1.69 (m, 5H), 1.69-1.83 (m, 1H), 2.69 (d, 2H), 3.20 (br s, 2H), 3.44 (br s, 2H), 7.42 (d, 1H), 7.85 (dd, 1H), 8.57 (s, 1H).

### EXAMPLE 65

### 6-(6-cyanohexyl)-N,N-diethylnicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M (6-cyano)hexylzinc bromide in THF (0.291 mmol). The mixture was heated in a single mode microwave synthesizer at 160° C. under nitrogen for 10 minutes, and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 288.3 (M+H)⁺; ¹H NMR (DMSO-d₆) δ 1.11 (br d, 6H), 1.29-1.44 (m, 4H), 1.51-1.60 (m, 2H), 1.64-1.75 (m, 2H), 2.47 (t, 2H), 2.81 (t, 2H), 3.20 (br s, 2H), 3.44 (br s, 2H), 7.45 (d, 1H), 7.85 (dd, 1H), 8.56 (s, 1H).

### EXAMPLE 66

### N,N-diethyl-6-(4-fluorobenzyl)nicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M 4-fluorobenzylzinc bromide in THF (0.291 mmol). The mixture was heated in a single mode microwave synthesizer at 160° C. under nitrogen for 10 minutes, and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 287.2 (M+H)⁺; ¹H NMR (DMSO-d₆) δ 1.10 (br d, 6H), 3.18 (br s, 2H), 3.43 (br s, 2H), 4.13 (s, 2H), 7.09-7.17 (m, 2H), 7.30-7.39 (m, 3H), 7.77 (dd, 1H), 8.51 (d, 1H).

### EXAMPLE 67

### methyl (3S)-3-{5-[(diethylamino)carbonyl]-2-pyridinyl}butanoate

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M 2R-(+)-3-methoxy-2-methyl-3-oxopropylzinc bromide bromide in THF (0.291 mmol). The mixture was heated in a single mode microwave synthesizer at 160° C. under nitrogen for 10 minutes, and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 279.2 (M+H)⁺; ¹H NMR (DMSO-d₆) δ 0.97-1.21 (br m, 9H), 2.84-2.92 (m, 1H), 2.97-3.05 (m, 1H), 3.08-3.15 (m, 1H), 3.18 (br s, 2H), 3.44 (br s, 2H), 3.56 (s, 3H), 7.32-7.41 (m, 1H), 7.74-7.81 (m, 1H), 8.50 (s, 1H).

### EXAMPLE 68

### 6-[(1S,2R,4R)-bicyclo[2.2.1]hept-2-yl]-N,N-diethylnicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M exo-2-norbornylzinc bromide solution in THF (0.291 mmol). The mixture was heated in a single mode microwave synthesizer at 160° C. under nitrogen for 10 minutes, and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 273.2 (M+H)⁺; ¹H NMR (DMSO-d₆) δ 0.96-1.32 (br m, 9H), 1.34-1.68 (m, 4H), 1.78-1.90 (m, 1H), 2.29-2.41 (m, 1.5H), 2.53-2.58 (m, 0.5H), 2.89-2.95 (m, 0.5H), 3.21(br s, 2H), 3.35-3.54 (br m, 2.5H), 7.43 (t, 1H), 7.75-7.86 (m, 1H), 8.54 (dd, 1H).

### EXAMPLE 69

### 6-cyclohexyl-N,N-diethylnicotinamide

A solution of 6-bromo-N,N-diethylnicotinamide (0.194 mmol) in THF (2.5 mL) was treated with [1,1′-bis(diphenylphosphino)ferrocene]dichloropalladium (0.010 mmol) and CuI (0.012 mmol) then treated with 0.5 M cyclohexylzinc bromide in THF (0.291 mmol). The mixture was heated in a single mode microwave synthesizer at 160° C. under nitrogen for 10 minutes, and concentrated in vacuo. The residue was dissolved in 1:1 DMSO/methanol and filtered through a membrane filter. The filtrate was concentrated in vacuo and the crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 261.2 (M+H)⁺; ¹H NMR (DMSO-d₆) δ 1.11 (br d, 6H), 1.19-1.30 (m, 1H), 1.31-1.44 (m, 2H), 1.46-1.59 (m, 2H), 1.67-1.75 (m, 1H), 1.77-1.93 (m, 4H), 2.72-2.81 (m, 1H), 3.20 (br s, 2H), 3.44 (br s, 2H), 7.45 (d, 1H), 7.86 (dd, 1H), 8.55 (d, 1H).

### EXAMPLE 70

### 6-methyl-N-propylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of propylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 179.1 (M+H)⁺; ¹H NMR (DMSO-d₆) δ 0.90 (t, 3H), 1.50-1.60 (m, 2H), 2.59 (s, 3H), 3.20-3.28 (m, 2H), 7.54 (d, 1H), 8.28 (dd, 1H), 8.65 (t, 1H), 8.95 (d, 1H).

### EXAMPLE 71

### N-isopropyl-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of isopropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 179.1 (M+H)⁺; ¹H NMR (DMSO-d₆) δ 1.18 (d, 6H), 2.59 (s, 3H), 4.05-4.15 (m, 1H), 7.54 (d, 1H), 8.29 (dd, 1H), 8.43 (d, 1H), 8.95 (d, 1H).

### EXAMPLE 72

### N-(sec-butyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of sec-butylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 193.2 (M+H)⁺; ¹H NMR (DMSO-d₆) δ 0.87 (t, 3H), 1.15 (d, 3H), 1.45-1.60 (m, 2H), 2.58 (s, 3H), 3.87-3.99 (m, 1H), 7.53 (d, 1H), 8.28 (dd, 1H), 8.35 (d, 1H), 8.95 (d, 1H).

### EXAMPLE 73

### N-isobutyl-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of isobutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 193.2 (M+H)⁺; ¹H NMR (DMSO-d₆) δ 0.90 (d, 6H), 1.79-1.90 (m, 1H), 2.61 (s, 3H), 3.08-3.13 (m, 2H), 7.59 (d, 1H), 8.34 (dd, 1H), 8.68 (t, 1H), 8.97 (d, 1H).

### EXAMPLE 74

### N-(tert-butyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of tert-butylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 193.2 (M+H)⁺; ¹H NMR (DMSO-d₆) δ 1.39 (s, 9H), 2.59 (s, 3H), 7.53 (d, 1H), 8.01 (s, 1H), 8.27 (dd, 1H), 8.92 (d, 11H).

### EXAMPLE 75

### 6-methyl-N-pentylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of n-pentylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 207.2 (M+H)⁺; ¹H NMR (DMSO-d₆) δ 0.86-0.92 (m, 3H), 1.26-1.36 (m, 4H), 1.49-1.58 (m, 2H), 2.60 (s, 3H), 3.24-3.30 (m, 2H), 7.58 (d, 1H), 8.32 (dd, 1H), 8.67 (t, 1H), 8.96 (d, 1H).

### EXAMPLE 76

### 6-methyl-N-(1-methylbutyl)nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-pentylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 207.2 (M+H)⁺; ¹H NMR (DMSO-d₆) δ0.88 (t, 3H), 1.14 (d, 3H), 1.24-1.38 (m, 2H), 1.39-1.58 (m, 2H), 2.60 (s, 3H), 3.98-4.08 (m, 1H), 7.55 (d, 1H), 8.31 (dd, 1H), 8.37 (d, 1H), 8.95 (d, 1H).

### EXAMPLE 77

### 6-methyl-N-(2-methylbutyl)nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-methylbutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 207.2 (M+H)⁺; ¹H NMR (DMSO-d₆) δ 0.84-0.93 (m, 6H), 1.08-1.19 (m, 1H), 1.37-1.47 (m, 1H), 1.60-1.70 (m, 1H), 2.60 (s, 3H), 3.05-3.13 (m, 1H), 3.18-3.26 (m, 1H), 7.56 (d, 1H), 8.30 (dd, 1H), 8.63 (t, 1H), 8.96 (d, 1H).

### EXAMPLE 78

### 6-methyl-N-(3-methylbutyl)nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3-methylbutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 207.2 (M+H)⁺; ¹H NMR (DMSO-d₆) δ 0.91 (d, 6H), 1.39-1.47 (m, 2H), 1.58-1.69 (m, 1H), 2.59 (s, 3H), 3.26-3.34 (m, 2H), 7.55 (d, 1H), 8.29 (dd, 1H), 8.62 (d, 1H), 8.94 (d, 1H).

### EXAMPLE 79

### 6-methyl-N-neopentylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2,2-dimethylpropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 207.2 (M+H)⁺; ¹H NMR (DMSO-d₆) δ 0.91 (s, 9H), 2.60 (s, 3H), 3.13 (d, 2H), 7.56 (d, 1H), 8.32 (dd, 1H), 8.54 (t, 1H), 8.97 (d, 1H).

### EXAMPLE 80

### N-(3,3-dimethylbutyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3,3-dimethylbutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 221.2 (M+H)⁺; ¹H NMR (DMSO-d₆) δ 0.93 (s, 9H), 1.42-1.52 (m, 2H), 2.58 (s, 3H), 3.26-3.34 (m, 2H), 7.53 (d, 1H), 8.26 (dd, 1H), 8.60 (t, 1H), 8.93 (d, 1H).

### EXAMPLE 81

### 6-methyl-N-(2,2,2-trifluoroethyl)nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2,2,2,-trifluoroethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 219.1 (M+H)⁺; ¹H NMR (DMSO-d₆) δ 2.58 (s, 3H), 4.06-4.20 (m, 2H), 7.51 (d, 1H), 8.25 (dd, 1H), 8.97 (d, 1H), 9.27 (t, 1H).

### EXAMPLE 82

### N-(2-methoxyethyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-methoxyethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 195.1 (M+H)⁺; ¹H NMR (DMSO-d₆) δ 2.58 (s, 3H), 3.27 (s, 3H), 3.42-3.49 (m, 4H), 7.51 (d, 1H), 8.26 (dd, 1H), 8.37 (t, 1H), 8.94 (d, 1H).

### EXAMPLE 83

### N-(2-methoxy-1-methylethyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 1-methyl-2-methoxyethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 209.1 (M+H)⁺; ¹H NMR (DMSO-d₆) δ 1.15 (d, 3H), 2.58 (s, 3H), 3.27 (s, 3H), 3.30 (q, 1H), 3.41 (q, 11H), 4.16-4.25 (m, 11H), 7.52 (d, 11H), 8.27 (dd, 1H), 4.44 (d, 1H), 8.94 (d, 11H).

### EXAMPLE 84

### N-(2-ethoxyethyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-ethoxyethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 209.1 (M+H)⁺; ¹H NMR (DMSO-d₆) δ 1.11 (t, 3H), 2.58 (s, 3H), 3.39-3.53 (m, 6H), 7.53 (d, 1H), 8.28 (dd, 1H), 8.74 (t, 1H), 8.95 (d, 1H).

### EXAMPLE 85

### N-(2-isopropoxyethyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-isopropoxyethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 223.2 (M+H)⁺; ¹H NMR (DMSO-d₆) δ 1.08 (d, 6H), 2.59 (s, 3H), 3.37-3.43 (m, 2H), 3.47-3.52 (m, 2H), 3.54-3.61 (m, 1H), 7.55 (d, 1H), 8.29 (dd, 1H), 8.72 (t, 1H), 8.95 (d, 11H).

### EXAMPLE 86

### 6-methyl-N-(3-propoxypropyl)nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3-propoxyethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 237.2 (M+H)⁺; ¹H NMR (DMSO-d₆) δ 0.86 (t, 3H), 1.45-1.56 (m, 2H), 1.73-1.81 (m, 2H), 2.59 (s, 3H), 3.29-3.37 (m, 4H), 3.39-3.46 (m, 2H), 7.55 (d, 1H), 8.28 (dd, 1H), 8.65 (t, 1H), 8.94 (d, 1H).

### EXAMPLE 87

### N-(3-methoxypropyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3-methoxypropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 209.1 (M+H)⁺; ¹H NMR (DMSO-d₆) δ 1.72-1.84 (m, 2H), 2.68 (s, 3H), 3.24 (s, 3H), 3.22 (q, 2H), 3.38 (t, 2H), 7.51 (d, 1H), 8.24 (dd, 1H), 8.64 (t, 1H), 8.93 (d, 1H).

### EXAMPLE 88

### 6-methyl-N-[(2S)-tetrahydro-2-furanylmethyl]nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (2S)-tetrahydro-2-furanylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 221.1 (M+H)⁺; ¹H NMR (DMSO-d₆) δ 1.53-1.63 (m, 1H), 1.74-1.98 (m, 3H), 2.59 (s, 3H), 3.34 (t, 2H), 3.61-3.66 (m, 1H), 3.75-3.81 (m, 1H), 3.95-4.01 (m, 1H), 7.55 (d, 1H), 8.31 (dd, 1H), 8.77 (t, 1H), 8.96 (d, 1H).

### EXAMPLE 89

### 6-methyl-N-[(2R)-tetrahydro-2-furanylmethyl]nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (2R)-tetrahydro-2-furanylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 221.1 (M+H)⁺; ¹H NMR (DMSO-d₆) δ 1.53-1.63 (m, 1H), 1.75-1.98 (m, 3H), 2.60 (s, 3H), 3.34 (t, 2H), 3.6-3.68 (m, 1H), 3.74-3.82 (m, 1H), 3.94-4.02 (m, 1H), 7.57 (d, 1H), 8.33 (dd, 1H), 8.79 (t, 1H), 8.97 (d, 1H).

### EXAMPLE 90

### 6-methyl-N-(tetrahydro-3-furanylmethyl)nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (2RS)-tetrahydro-2-furanylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 221.1 (M+H)⁺; ¹H NMR (DMSO-d₆) δ 1.56-1.65 (m, 1H), 1.91-2.00 (m, 1H), 2.44-2.53 (m, 1H), 2.60 (s, 3H), 3.21-3.33 (m, 2H), 3.48 (q, 1H), 3.59-3.65 (m, 1H), 3.69 (q, 1H), 3.72-3.78 (m, 1H), 7.57 (d, 1H), 8.31 (dd, 1H), 8.79 (t, 1H), 8.96 (d, 11H).

### EXAMPLE 91

### N-(cyanomethyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of aminoacetonitrile (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 176.1 (M+H)⁺; ¹H NMR (DMSO-d₆) δ 2.59 (s, 3H), 4.07-4.10 (m, 2H), 7.49 (d, 1H), 8.41 (dd, 11H), 8.94 (d, 11H), 9.37 (t, 11H).

### EXAMPLE 92

### N-cyclopropyl-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclopropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 177.1 (M+H)⁺; ¹H NMR (DMSO-d₆) δ 0.53-0.63 (m, 2H), 0.67-0.76 (m, 2H), 2.57 (s, 3H), 2.82-2.91 (m, 1H), 7.49 (d, 1H), 8.21 (dd, 1H), 8.62 (d, 1H), 8.90 (d, 1H).

### EXAMPLE 93

### N-(cyclopropylmethyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclopropylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 191.1 (M+H)⁺; ¹H NMR (DMSO-d₆) 60.20-0.29 (m, 2H), 0.39-0.51 (m, 2H), 0.97-1.08 (m, 1H), 2.57 (s, 3H), 3.12-3.20 (m, 2H), 7.52 (d, 1H), 8.27 (dd, 1H), 8.75 (t, 1H), 8.95 (d, 1H).

### EXAMPLE 94

### N-cyclobutyl-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclobutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 191.1 (M+H)⁺; ¹H NMR (DMSO-d₆) δ 1.68-1.78 (m, 2H), 2.00-2.12 (m, 2H), 2.19-2.28 (m, 2H), 2.68 (s, 3H), 4.36-4.49 (m, 1H), 7.53 (d, 1H), 8.28 (dd, 1H), 8.80 (d, 1H), 8.95 (d, 1H).

### EXAMPLE 95

### N-cyclopentyl-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclopentylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 205.2 (M+H)⁺; ¹H NMR (DMSO-d₆) δ 1.48-1.60 (m, 4H), 1.64-1.75 (m, 2H), 1.85-1.97 (m, 2H), 2.59 (s, 3H), 4.18-4.28 (m 1H), 7.53 (d, 1H), 8.28 (dd, 1H), 8.48 (d, 1H), 8.94 (d, 1H).

### EXAMPLE 96

### N-(cyclopentylmethyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclopentylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 219.2 (M+H)⁺; ¹H NMR (DMSO-d₆) δ 1.20-1.31 (m, 2H), 1.45-1.64 (m, 4H), 1.64-1.74 (m, 2H), 2.09-2.19 (m, 1H), 2.59 (s, 3H), 3.18-3.24 (m, 2H), 7.55 (d, 1H), 8.29 (dd, 1H), 8.67 (t, 1H), 8.95 (d, 1H).

### EXAMPLE 97

### N-cyclohexyl-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclohexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 219.2 (M+H)⁺; ¹H NMR (DMSO-d₆) δ 1.07-1.20 (m, 1H), 1.25-1.37 (m, 4H), 1.57-1.65 (m, 1H), 1.68-1.79 (m, 2H), 1.79-1.89 (m, 2H), 2.57 (s, 3H), 3.73-3.81 (m, 1H), 7.50 (d, 1H), 8.25 (dd, 1H), 8.39 (d, 1H), 8.93 (d, 1H).

### EXAMPLE 98

### 6-methyl-N-(2-methylcyclohexyl)nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-methylcyclohexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 233.2 (M+H)⁺; ¹H NMR (DMSO-d₆) δ 0.87 (dd, 3H), 0.98-1.13 (m, 1H), 1.13-1.41 (m, 3H), 1.42-1.58 (m, 2H), 1.59-1.86 (m, 3H), 2.59 (s, 3H), 3.45-3.55 (m, 1H), 7.51-7.57 (m, 1H), 8.27-8.32 (m, 1H), 8.37 (d, 1H), 8.93-8.96 (m, 1H).

### EXAMPLE 99

### 6-methyl-N-(4-methylcyclohexyl)nicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 4-methylcyclohexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 233.2 (M+H)⁺; ¹H NMR (DMSO-d₆) δ 0.89 (d, 1.5H), 0.94 (d, 1.5H), 0.97-1.08 (m, 1H), 1.29-1.46 (br m, 2.5H), 1.47-1.75 (br m, 4.5H), 1.81-0.189 (m, 1H), 2.61 (d, 3H), 3.68-3.77 (m, 0.5H), 3.89-3.96 (m, 0.5H), 7.58 (t, 1H), 8.30-8.36 (m, 1H), 8.43 (d, 1H), 8.96 (t, 1H).

### EXAMPLE 100

### N-cycloheptyl-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cycloheptylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 233.2 (M+H)⁺; ¹H NMR (DMSO-d₆) δ 1.34-1.75 (m, 10OH), 1.82-1.93 (m, 2H), 2.59 (s, 3H), 3.91-4.01 (m, 1H), 7.54 (d, 1H), 8.30 (dd, 1H), 8.46 (d, 1H), 8.94 (d, 1H).

### EXAMPLE 101

### N-{[(1S,2R,5S)-6,6-dimethylbicyclo[3.1.1]hept-2-yl]methyl}-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (−)-cis-myrtanylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 273.2 (M+H)⁺; ¹H NMR (DMSO-d₆) δ 0.86 (d, 11H), 1.07 (s, 3H), 1.18 (s, 3H), 1.47-1.57 (m, 1H), 1.78-1.97 (m, 5H), 2.25-2.38 (m, 2H), 2.59 (s, 3H), 3.26-3.33 (m, 2H), 7.55 (d, 1H), 8.28 (dd, 1H), 8.64 (t, 1H), 8.94 (d, 1H).

### EXAMPLE 102

### N-(1-adamantylmethyl)-6-methylnicotinamide

A suspension of 6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 1-adamantylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 285.2 (M+H)⁺; ¹H NMR (DMSO-d₆) δ 1.50 (d, 6H), 1.63 (q, 6H), 1.93 (br s, 3H), 2.59 (s, 3H), 3.01 (d, 2H), 7.57 (d, 1H), 8.33 (dd, 1H), 8.50 (t, 1H), 8.97 (d, 1H).

### EXAMPLE 103

### N-isopropyl-N,2-dimethyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-isopropyl-N-methylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 261.2 (M+H)⁺.

### EXAMPLE 104

### N-butyl-N,2-dimethyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-butyl-N-methylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 275.1 (M+H)⁺.

### EXAMPLE 105

### N-isobutyl-N,2-dimethyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-isobutyl-N-methylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 275.1 (M+H)⁺.

### EXAMPLE 106

### N-(1,3-dioxolan-2-ylmethyl)-N,2-dimethyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-(1,3-dioxolan-2-ylmethyl)-N-methylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 305.1 (M+H)⁺; ¹H NMR (DMSO-d₆) δ 2.55 (d, 3H), 2.95 (s, 1.5H), 3.21 (s, 1.5H), 3.38 (br s, 1H), 3.76 (br s, 1H), 3.80-3.89 (m,2H), 3.89-3.94 (m, 1H), 4.01-4.06 (m, 1H), 4.92 (t, 0.5H), 5.18 (t, 0.5H), 7.69 (q, 1H), 7.87 (q, 1H).

### EXAMPLE 107

### N,2-dimethyl-N-2-propynyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-methyl-N-propargylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 257.0 (M+H)⁺; ¹H NMR (DMSO-d₆) δ 2.55 (d, 3H), 2.76-2.78 (m, 0.5H), 2.85-2.87 (m, 0.5H), 2.93 (s, 1.5H), 3.20 (s, 1.5H), 3.96 (br s, 1H), 4.43 (br s, 1H), 7.72 (dd, 1H), 7.88 (q, 1H).

### EXAMPLE 108

### N-cyclohexyl-N,2-dimethyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-cyclohexyl-N-methylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 301.1 (M+H)⁺; ¹H NMR (DMSO-d₆) δ 1.06-1.13 (br m, 1H), 1.19-1.33 (br m, 1H), 1.44-1.52 (m, 1H), 1.59-1.64 (m, 1H). 1.66-1.83 (br m, 5H), 1.87-1.93 (br m, 1H), 2.53 (d, 3H), 2.73 (s, 1.5H), 3.04 (s, 1.5H), 4.45-4.53 (m, 1H), 7.70 (dd, 1H), 7.83 (t, 1H).

### EXAMPLE 109

### N-ethyl-2-methyl-N-propyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-ethyl-N-propylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 275.1 (M+H)⁺.

### EXAMPLE 110

### N-butyl-N-isopropyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-butyl-N-isopropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 303.1 (M+H)⁺.

### EXAMPLE 111

### N-cyclohexyl-N-ethyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-cyclohexyl-N-ethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 315.1 (M+H)⁺.

### EXAMPLE 112

### N-isopropyl-2-methyl-N-propyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-isopropyl-N-propylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 289.1 (M+H)⁺.

### EXAMPLE 113

### N-butyl-2-methyl-N-propyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-butyl-N-propylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 303.1 (M+H)⁺.

### EXAMPLE 114

### N-(cyanomethyl)-N,2-dimethyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-cyanomethyl-N-methylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 258.0 (M+H)⁺; ¹H NMR (DMSO-d₆) δ 2.56 (s, 3H), 2.98 (s, 2.5H), 3.24 (s, 0.5H), 4.62 (s, 2H), 7.73 (d, 1H), 7.92 (d, 1H).

### EXAMPLE 115

### N,N-dibutyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N,N-dibutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS n/e 317.1 (M+H)⁺; ¹H NMR (DMSO-d₆) δ 0.77 (t, 3H), 1.01 (t, 3H), 1.10-1.19 (m, 2H), 1.39-1.55 (br m, 2H), 1.66-1.75 (m, 2H), 2.54 (s, 3H), 3.12 (br s, 2H), 3.57 (br s, 2H), 7.70 (d, 1H), 7.85 (d, 1H).

### EXAMPLE 116

### N,N-diisobutyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N,N-diisobutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 317.1 (M+H)⁺; ¹H NMR (DMSO-d₆) δ 0.79 (d, 6H), 1.03 (d, 6H), 1.83-1.92 (m, 1H), 2.14-2.22 (m, 1H), 2.57 (s, 3H), 3.04 (br s, 2H), 3.44 (br s, 2H), 7.70 (d, 1H), 7.84 (d, 1H).

### EXAMPLE 117

### N-(2-cyanoethyl)-N,2-dimethyl-6-(trifluoromethyl)nicotinamide

A suspension of 2-methyl-6-(trifluoromethyl)nicotinic acid (6 mmol) in dry dicbloromethane (9 mL) was treated with thionyl chloride (12.4 mmol) at 0° C., stirred for one hour, and concentrated in vacuo. The concentrate was added dropwise to a cold solution of N-cyanoethyl-N-methylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 272.0 (M+H)⁺; ¹H NMR (DMSO-d₆) δ 2.54-2.57 (m, 1.5H), 2.59 (s, 1.5H), 2.90-2.93 (m, 1.5H), 2.95 (s, 1.5H), 3.12-3.22 (m, 2H), 3.83-3.90 (m, 1H), 4.52 (br s, 1H), 7.72 (d, 1H), 7.89 (d, 1H).

### EXAMPLE 118

### N-butyl-N-(cyanomethyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of N-butyl-N-cyanomethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 300.1 (M+H)⁺; ¹H NMR (DMSO-d₆) δ 0.81 (t, 2H), 1.03 (t, 1H), 1.15-1.24 (m, 1.5H), 1.41-1.50 (m, 0.5H), 1.54-1.63 (m, 1.5H), 1.71-1.80 (m, 0.5H), 2.56 (s, 3H), 3.26 (t, 1.5H), 3.69 (t, 0.5H), 4.29 (s, 0.5H), 4.58 (s, 1.5H), 7.74 (t, 1H), 7.90-7.95 (m, 1H).

### EXAMPLE 119

### N-(sec-butyl)-2-chloro-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of sec-butylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 227.1 (M+H)⁺; ¹H NMR (DMSO-d₆) δ 0.90 (t, 3H), 1.11 (d, 3H), 1.43-1.51 (m, 2H), 2.47 (s, 3H), 3.80-3.83 (m, 1H), 7.31 (d, 1H), 7.72 (d, 1H), 8.26 (d, 1H).

### EXAMPLE 120

### 2-chloro-6-methyl-N-pentylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of pentylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 241.0 (M+H)⁺; ¹H NMR (DMSO-d₆) δ 0.85-0.92 (m, 3H), 1.27-1.36 (m, 4H), 1.45-1.56 (m, 2H), 2.47 (s, 3H), 3.17-3.24 (m, 2H), 7.32 (d, 1H), 7.73 (d, 1H), 8.43 (t, 1H).

### EXAMPLE 121

### 2-chloro-6-methyl-N-(2-methylbutyl)nicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-methylbutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 241.0 (M+H)⁺; ¹H NMR (DMSO-d₆) 0.82-0.95 (m, 6H), 1.09-1.21 (m, 1H), 1.37-1.49 (m, 1H), 1.54-1.66 (m, 1H), 2.47 (s, 3H), 2.99-3.08 (m, 1H), 3.11-3.19 (m, 1H), 7.32 (d, 1H), 7.73 (s, 1H). 8.43 (t, 1H).

### EXAMPLE 122

### 2-chloro-N-(2-ethoxyethyl)-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-ethoxyethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 243.0 (M+H)⁺; ¹H NMR (DMSO-d₆) δ 1.12 (t, 3H), 2.47 (s, 3H), 3.37 (q, 2H), 3.43-3.51 (m, 4H), 7.32 (d, 1H), 7.73 (d, 1H), 8.53 (t, 1H).

### EXAMPLE 123

### 2-chloro-6-methyl-N-(3-propoxypropyl)nicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3-propoxypropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 271.0 (M+H)⁺; ¹H NMR (DMSO-d₆) δ 0.86 (t, 3H), 1.36-1.46 (m, 2H), 1.68-1.77 (m, 2H), 2.47 (s, 3H), 3.24-3.34 (m, 4H), 3.43 (t, 2H), 7.32 (d, 1H), 7.74 (d, 1H), 8.44 (t, 1H).

### EXAMPLE 124

### 2-chloro-N-(3-methoxypropyl)-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3-methoxypropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 243.0 (M+H)⁺; ¹H NMR (DMSO-d₆) δ 1.68-1.77 (m, 2H), 2.47 (s, 3H), 3.24 (s, 3H), 3.24-3.29 (m, 2H), 3.39 (t, 2H), 7.32 (d, 1H), 7.74 (d, 1H), 8.46 (t, 1H).

### EXAMPLE 125

### 2-chloro-6-methyl-N-[(2S)-tetrahydro-2-furanylmethyl]nicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (2S)-tetrahydro-2-furanylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 255.0 (M+H)⁺; ¹H NMR (DMSO-d₆) δ 1.56-1.67 (m, 1H), 1.76-1.99 (m, 3H), 2.47 (s, 3H), 3.22-3.35 (m, 2H), 3.59-3.66 (m, 1H), 3.74-3.81 (m, 1H), 3.90-3.97 (m, 1H), 7.32 (d, 1H), 7.72 (d, 1H), 8.55 (t, 1H).

### EXAMPLE 126

### 2-chloro-6-methyl-N-[(2R)-tetrahydro-2-furanylmethyllnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (2R)-tetrahydro-2-furanylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 255.0 (M+H)⁺; ¹H NMR (DMSO-d₆) δ 1.55-1.67 (m, 1H), 1.77-1.97 (m, 3H), 2.47 (s, 3H), 3.21-3.36 (m, 2H), 3.59-3.68 (m, 1H), 3.74-3.81 (m, 1H), 3.90-3.97 (m, 1H), 7.32 (d, 1H), 7.72 (d, 1H), 8.55 (t, 1H).

### EXAMPLE 127

### 2-chloro-N-(cyanomethyl)-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of aminoacetonitrile hydrochloride (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 210 (M+H)⁺; ¹H NMR (DMSO-d₆) δ 2.49 (s, 3H), 4.33 (d, 2H), 7.37 (d, 1H), 7.83 (d, 1H), 9.25 (t, 1H).

### EXAMPLE 128

### 2-chloro-N-cyclopropyl-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclopropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 211 (M+H)⁺; ¹H NMR (DMSO-d₆) δ 0.49-0.54 (m, 2H), 0.67-0.72 (m, 2H), 2.47 (s, 3H), 2.76-2.84 (m, 1H), 7.31 (d, 1H), 7.73 (d, 1H), 8.50 (d, 1H).

### EXAMPLE 129

### 2-chloro-N-(cyclopropylmethyl)-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclopropylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 225 (M+H)⁺; ¹H NMR (DMSO-d₆) δ 0.19-0.26 (m, 2H), 0.40-0.48 (m, 2H), 0.94-1.04 (m, 1H), 2.47 (s, 3H), 3.12 (t, 2H), 7.32 (d, 1H), 7.74 (d, 1H), 8.54 (t, 1H).

### EXAMPLE 130

### 2-chloro-N-cyclohexyl-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclohexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 253 (M+H)⁺; ¹H NMR (DMSO-d₆) δ 1.08-1.36 (m, 5H), 1.53-1.61 (m, 1H), 1.67-1.76 (m, 2H), 1.79-1.87 (m, 2H), 2.47 (s, 3H), 3.65-3.75 (m, 1H), 7.31 (d, 1H), 7.71 (d, 1H), 8.33 (d, 1H).

### EXAMPLE 131

### 2-chloro-6-methyl-N-(3-methylcyclohexyl)nicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3-methylcyclohexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 267 (M+H)⁺; ¹H NMR (DMSO-d₆) δ 0.73-0.84 (m, 0.65H), 0.85-0.93 (m, 3.35H), 0.97-1.05 (m, 0.35H), 1.06-1.16 (m, 0.65H), 1.22-1.37 (m, 1H), 1.39-1.75(m, 4H), 1.77-1.89 (m, 2H), 2.47 (d, 3H), 3.64-3.74 (m, 0.65H), 4.07 (br s, 0.35H), 7.30 (d, 1H), 7.71 (d, 1H), 8.32 (d, 1H).

### EXAMPLE 132

### N-(4-tert-butylcyclohexyl)-2-chloro-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 4-tert-butylcyclohexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 309 (M+H)⁺; ¹H NMR (DMSO-d₆) δ 0.80-0.87 (m, 9H), 0.91-1.13 (m, 2.5H), 1.15-1.27 (m, 1.5H), 1.27-1.38 (m, 0.5H), 1.42-1.56 (m, 1H), 1.72-1.80 (m, 1.5H), 1.83-1.90 (m, 0.5H), 1.90-1.96 (m, 1.5H), 2.47 (d, 3H), 3.57-3.66 (m, 0.7H), 4.05 (br s, 0.3H), 7.29-7.33 (m, 1H), 7.69-7.73 (m, 1H), 8.29-8.35 (m, 1H).

### EXAMPLE 133

### 2-chloro-N-(cyclohexylmethyl)-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclohexylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 267 (M+H)⁺; ¹H NMR (DMSO-d₆) δ 0.87-1.00 (m, 2H), 1.07-1.26 (m, 3H), 1.44-1.56 (m, 1H), 1.57-1.79 (m, 5H), 2.47 (s, 3H), 3.06 (t, 2H), 7.31 (d, 1H), 7.73 (d, 1H), 8.43 (t, 1H).

### EXAMPLE 134

### 2-chloro-N-cycloheptyl-6-methylnicotinamide

A suspension of 2-chloro-6-methylnicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cycloheptylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 267 (M+H)⁺; ¹H NMR (DMSO-d₆) δ 1.37-1.69 (m, 10H), 1.81-1.92 (m, 2H), 2.47 (s, 3H), 3.84-3.95 (m, 1H), 7.30 (d, 1H), 7.70 (d, 1H), 8.38 (d, 1H).

### EXAMPLE 135

### N-ethyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of ethylamine hydrochloride (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 233 (M+H)⁺; ¹H NMR (DMSO-d₆) δ 1.13 (t, 3H), 2.57 (s, 3H), 3.25-3.32 (m, 2H), 7.78 (d, 11H), 7.96 (d, 11H), 8.55 (br t, 1H).

### EXAMPLE 136

### 2-methyl-N-propyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of propylamine hydrochloride (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 247 (M+H)⁺; ¹H NMR (DMSO-d₆) δ 0.92 (t, 3H), 1.49-1.58 (m, 2H), 2.57 (s, 3H), 3.22 (q, 2H), 7.78 (d, 1H), 7.95 (d, 1H), 8.55 (br t, 1H).

### EXAMPLE 137

### N-isopropyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of isopropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acelonitrile/water containing 0.01% TFA over 50 minutes to provide the desired product as the trifluoroacetate salt. MS m/e 247 (M+H)⁺; ¹H NMR (DMSO-d₆) δ 1.16 (d, 6H), 2.56 (s, 3H), 4.02-4.10 (m, 1H), 7.77 (d, 1H), 7.93 (d, 1H), 8.43 (d, 1H).

### EXAMPLE 138

### N-butyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of butylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 261 (M+H)⁺; ¹H NMR (DMSO-d₆) δ 0.91 (t, 3H), 1.31-1.40 (m, 2H), 1.47-1.55 (m, 2H), 2.57 (s, 3H), 3.26 (q, 2H), 7.78 (d, 1H), 7.95 (d, 1H), 8.54 (br t, 1H).

### EXAMPLE 139

### N-(sec-butyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of sec-butylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 261 (M+H)⁺; ¹H NMR (DMSO-d₆) δ 0.91 (t, 3H), 1.14 (d, 3H), 1.45-1.54 (m, 2H), 2.57 (s, 3H), 3.85-3.94 (m, 1H), 7.78 (d, 1H), 7.93 (d, 1H), 8.37 (d, 1H).

### EXAMPLE 140

### N-isobutyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of isobutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 261 (M+H)⁺; ¹H NMR (DMSO-d₆) δ 0.92 (d, 6H), 1.78-1.87 (m, 1H), 2.57 (s, 3H), 3.10 (t, 2H), 7.79 (d, 1H), 7.95 (d, 1H), 8.56 (br t, 1H).

### EXAMPLE 141

### N-(tert-butyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of tert-butylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 261 (M+H)⁺; ¹H NMR (DMSO-d₆) δ 1.37 (s, 9H), 2.57 (s, 3H), 7.74 (d, 1H), 7.88 (d, 1H), 8.16 (br s, 1H).

### EXAMPLE 142

### 2-methyl-N-pentyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of pentylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 275 (M+H)⁺; ¹H NMR (DMSO-d₆) δ 0.86-0.91 (m, 3H), 1.29-1.36 (m, 4H), 1.48-1.56 (m, 2H), 2.57 (s, 3H), 3.23-3.29 (m, 2H), 7.78 (d, 1H), 7.94 (d, 1H), 8.54 (brt, 1H).

### EXAMPLE 143

### 2-methyl-N-(1-methylbutyl)-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-pentylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 275 (M+H)⁺; ¹H NMR (DMSO-d₆) δ 0.91 (t, 3H), 1.14 (d, 3H), 1.29-1.52 (m, 4H), 2.57 (s, 3H), 3.93-4.03 (m, 1H), 7.77 (d, 1H), 7.92 (d, 1H), 8.37 (d, 1H).

### EXAMPLE 144

### 2-methyl-N-(2-methylbutyl)-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (2-methyl)butylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 275 (M+H)⁺; ¹H NMR (DMSO-d₆) δ 0.86-0.93 (m, 6H), 1.12-1.20 (m, 1H), 1.37-1.47 (m, 1H), 1.57-1.66 (m, 1H), 2.57 (s, 3H), 3.05-3.13 (m, 1H), 3.17-3.24 (m, 11H), 7.78 (d, 11H), 7.94 (d, 11H), 8.54 (t, 1H).

### EXAMPLE 145

### 2-methyl-N-(3-methylbutyl)-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (3-methyl)butylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 275 (M+H)⁺; ¹H NMR (DMSO-d₆) δ 0.91 (d, 6H), 1.42 (q, 2H), 1.61-1.70 (m, 1H), 2.56 (s, 3H), 3.24-3.29 (m, 2H), 7.78 (d, 1H), 7.94 (d, 1H), 8.52 (brt, 1H).

### EXAMPLE 146

### N-(1,1-dimethylpropyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (1,1-dimethyl)propylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 275 (M+H)⁺; ¹H NMR (DMSO-d₆) δ 0.86 (t, 3H), 1.31 (s, 6H), 1.76 (q, 2H), 2.56 (s, 3H), 7.75 (d, 1H), 7.87 (d, 1H), 8.01 (s, 1H).

### EXAMPLE 147

### N-(1-ethylpropyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3-pentylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acelonitrile/water containing 0.01% TFA over 50 minutes to provide the desired product as the trifluoroacetate salt. MS m/e 275 (M+H)⁺; ¹H NMR (DMSO-d₆) δ 0.91 (t, 6H), 1.36-1.47 (m, 2H), 1.50-1.60 (m, 2H), 2.58 (s, 3H), 3.71-3.81 (m, 1H), 7.78 (d, 1H), 7.92 (d, 1H), 8.28 (d, 1H).

### EXAMPLE 148

### N-hexyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of hexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 289 (M+H)⁺; ¹H NMR (DMSO-d₆) δ 0.88 (t, 3H), 1.26-1.37 (m, 6H), 1.47-1.56 (m, 2H), 2.57 (s, 3H), 3.22-3.29 (m, 2H), 7.78 (d, 1H), 7.94 (d, 1H), 8.54 (br t, 1H).

### EXAMPLE 149

### N-(3,3-dimethylbutyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3,3-dimethylbutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 289 (M+H)⁺; ¹H NMR (DMSO-d₆) δ 0.88 (t, 3H), 1.26-1.37 (m, 6H), 1.47-1.56 (m, 2H), 2.57 (s, 3H), 3.22-3.29 (m, 2H), 7.78 (d, 1H), 7.94 (d, 1H), 8.54 (br t, 1H).

### EXAMPLE 150

### N-(2-methoxy-1-methylethyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-methoxy-1-methylethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 277 (M+H)⁺; ¹H NMR (DMSO-d₆) δ 1.13 (d, 3H), 1.22-1.27 (m, 2H), 2.54 (s, 3H), 2.57 (s, 3H), 3.36-3.40 (m, 1H), 7.78 (d, 1H), 7.92 (d, 1H), 8.46 (d, 1H).

### EXAMPLE 151

### 2-methyl-N-[2-(methylsulfanyl)ethyl]-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-methylthioethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 279 (M+H)⁺; ¹H NMR (DMSO-d₆) δ 2.11 (s, 3H), 2.60 (s, 3H), 2.67 (t, 2H), 3.46 (q, 2H), 7.80 (d, 11H), 7.97 (d, 1H), 8.70 (br t, 1H).

### EXAMPLE 152

### N-(2-isopropoxyethyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-isopropoxyethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 291.1 (M+H)⁺; ¹H NMR (DMSO-d₆) δ 1.10 (d, 6H), 2.57 (s, 3H), 3.39 (q, 2H), 3.50 (t, 2H), 3.55-3.63 (m, 1H), 7.79 (d, 1H), 7.93 (d, 1H), 8.60 (br t, 1H).

### EXAMPLE 153

### 2-methyl-N-(3-propoxypropyl)-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3-propoxypropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 305.1 (M+H)⁺; ¹H NMR (DMSO-d₆) δ 0.87 (t, 3H), 1.47-1.55 (m, 2H), 1.72-1.79 (m, 2H), 2.57 (s, 3H), 3.28-3.34 (m, 4H), 3.43 (t, 2H), 7.78 (d, 1H), 7.96 (d, 1H), 8.54 (br t, 1H).

### EXAMPLE 154

### N-(3-methoxypropyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 3-methoxypropylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 277.0 (M+H)⁺; ¹H NMR (DMSO-d₆) δ 1.72-1.79 (m, 2H), 2.57 (s, 3H), 3.24 (s, 3H), 3.27-3.33 (m, 2H), 3.39 (t, 2H), 7.79 (d, 1H), 7.96 (d, 1H), 8.56 (br t, 11H).

### EXAMPLE 155

### 2-methyl-N-[(2S)-tetrahydro-2-furanylmethyl]-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (2S)-tetrahydro-2-furanylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 289.0 (M+H)⁺; ¹H NMR (DMSO-d₆) δ 1.55-1.64(m, 1H), 1.78-1.99 (m, 3H), 2.57 (s, 3H), 3.29-3.35 (m, 2H), 3.64 (q, 1H), 3.78 (q, 1H), 3.94-4.00 (m, 1H), 7.78 (d, 1H), 7.94 (d, 1H), 8.66 (br t, 1H).

### EXAMPLE 156

### 2-methyl-N-[(2R)-tetrahydro-2-furanylmethyl]-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of (2R)-tetrahydro-2-furanylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 289.0 (M+H)⁺; ¹H NMR (DMSO-d₆) δ 1.55-1.63 (m, 1H), 1.77-1.99 (m, 3H), 2.57 (s, 3H), 3.29-3.35 (m, 2H), 3.64 (q, 1H), 3.78 (q, 1H), 3.94-4.00 (m, 1H), 7.78 (d, 1H), 7.94 (d, 1H), 8.66 (br t, 1H).

### EXAMPLE 157

### N-(cyanomethyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of aminoacetonitrile (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 244.0 (M+H)⁺; ¹H NMR (DMSO-d₆) δ 2.59 (s, 3H), 4.37 (d, 2H), 7.83 (d, 11H), 8.05 (d, 1H), 9.35 (br t, 1H).

### EXAMPLE 158

### 2-methyl-N-2-propynyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of propargylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 243.2 (M+H)⁺; ¹H NMR (DMSO-d₆) δ 2.58 (s, 3H), 3.18 (t, 1H), 4.08 (q, 2H), 7.80 (d, 1H), 7.98 (d, 1H), 9.05 (br t, 1H).

### EXAMPLE 159

### N-(cyclopropylmethyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of aminomethylcyclopropane (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 259.0 (M+H)⁺; ¹H NMR (DMSO-d₆) δ 0.21-0.27 (m, 2H), 0.43-0.49 (m, 2H), 0.97-1.07 (m, 1H), 2.58 (s, 3H), 3.16 (t, 2H), 7.78 (d, 1H), 7.95 (d, 1H), 8.67 (br t, 1H).

### EXAMPLE 160

### N-cyclobutyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclobutylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 259.0 (M+H)⁺; ¹H NMR (DMSO-d₆) δ 1.64-1.75 (m, 2H), 1.95-2.06 (m, 2H), 2.20-2.31 (m, 2H), 2.56 (s, 3H), 4.33-4.43 (m, 1H), 7.78 (d, 1H), 7.96 (d, 1H), 8.79 (d, 1H).

### EXAMPLE 161

### N-cyclopentyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclopentylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 273.1 (M+H)⁺; ¹H NMR (DMSO-d₆) δ 1.47-1.60 (m, 4H), 1.61-1.72 (m, 2H), 1.84-1.94 (m, 2H), 2.56 (s, 3H), 4.17-4.25 (m, 1H), 7.77 (d, 1H), 7.93 (d, 1H), 8.51 (d, 1H).

### EXAMPLE 162

### N-cyclohexyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of cyclohexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 287.1 (M+H)⁺; ¹H NMR (DMSO-d₆) δ 1.10-1.37 (m, 5H), 1.54-1.62 (m, 1H), 1.69-1.76 (m, 2H), 1.82-1.89 (m, 2H), 2.56 (s, 3H), 3.71-3.80 (m, 1H), 7.77 (d, 1H), 7.92 (d, 1H), 8.43 (d, 11H).

### EXAMPLE 163

### 2-methyl-N-(2-methylcyclohexyl)-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-methylcyclohexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 301.1 (M+H)⁺; ¹H NMR (DMSO-d₆) δ 0.89 (d, 1H), 0.93 (d, 2H), 1.00-1.11 (m, 0.7H), 1.12-1.33 (br m, 2.3H), 1.33-1.45 (br m, 1.5H), 1.46-1.78 (br m, 3.5H), 1.87 (br d, 1H), 2.56 (s, 1H), 2.57 (s, 2H), 3.41-3.50 (m, 0.65H), 4.07-4.14 (m, 0.35H), 7.75-7.81 (m, 1H), 7.87-7.81 (m, 1H), 8.30 (d, 0.35H), 8.36 (d, 0.65H).

### EXAMPLE 164

### 2-methyl-N-(4-methylcyclohexyl)-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 4-methylcyclohexylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 301.1 (M+H)⁺; ¹H NMR (DMSO-d₆) δ 0.89 (q, 3H), 0.97-1.07 (m, 1H), 1.21-1.37 (br m, 2.5H), 1.45-1.61 (br m, 2.5H), 1.64-1.73 (m, 2H), 1.85-1.92 (m, 1H), 2.56 (s, 3H), 3.64-3.72 (m, 0.5H), 3.98-4.05 (m, 0.5H), 7.77 (d, 1H), 7.92 (d, 1H), 8.41 (d, 1H).

### EXAMPLE 165

### N-2-adamantyl-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 2-adamantanamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 339.1 (M+H)⁺; ¹H NMR (DMSO-d₆) δ 1.53 (br d, 2H), 1.71 (br s, 2H), 1.83 (br t, 6H), 1.95 (br s, 2H), 2.03 (br d, 2H), 2.56 (s, 3H), 4.06-4.10 (br m, 1H), 7.76 (d, 1H), 7.92 (d, 1H), 8.47 (d, 1H).

### EXAMPLE 166

### N-(1-adamantylmethyl)-2-methyl-6-(trifluoromethyl)nicotinamide

A suspension of 2-methyl-6-(trifluoromethyl)nicotinic acid (6 mmol) in dry dichloromethane (9 mL) was treated with thionyl chloride (12.4 mmol) at 0° C., stirred for one hour, and concentrated in vacuo. The concentrate was added dropwise to a cold solution of 1-adamantylmethylamine (6 mmol) and triethylamine (4.5 mL) in dichloromethane (20 mL). The mixture was stirred for 4 hours and then concentrated in vacuo. The residue was dissolved in dichloromethane, washed sequentially with saturated sodium bicarbonate, water, and brine, dried (MgSO₄), filtered, and concentrated in vacuo. The crude product was purified by HPLC on a C-18 column using a solvent system increasing in gradient from 5% to 100% acetonitrile/water containing 0.01% TFA over 50 minutes to provide the desired product as the trifluoroacetate salt. MS m/e 353.1 (M+H)⁺; ¹H NMR (DMSO-d₆) δ 1.52 (br d, 6H), 1.58-1.71 (br m, 6H), 1.95 (br s, 3H), 2.58 (s, 3H), 2.98 (d, 2H), 7.78 (d, 1H), 7.95 (d, 1H), 8.44 (br t,1H).

### EXAMPLE 167

### 6-(diethylamino)-N,N-diethylnicotinamide

A solution of 6-chloro-N,N-diethylnicotinamide (0.213 g, 1.0 mmol), N,N-diethylamine (0.696 mL, 5.0 mmol), and triethylamine (0.696 mL, 5.0 mmol) in N-methylpyrrolidinone (5 mL) was heated to 150° C. for 24 hours and concentrated in vacuo. The residue was purified by HPLC using a C-18 column and a solvent mixture varying in gradient from 10% to 50% acetonitrile/water containing 0.01% TFA over 50 minutes. The pure fractions were lyophilized to provide the desired product as the trifluoroacetate salt. This was dissolved in dichloromethane and shaken with trisamine resin (substitution 4.42 mmol/g, 2.2 mmol). The resin was filtered and the filtrate was concentrated in vacuo. The residue was dissolved in diethyl ether treated with 2 M HCl in diethyl ether (2 mL, 4.0 mmol). The precipitate was filtered and crystallized from methanol/ethyl acetate/hexanes to provide the desired product as the dihydrochloride salt. MS m/e 250.1 (M+H)⁺; ¹H NMR (DMSO-d₆) δ 1.09-1.16 (m, 12H), 3.35 (q, 4H), 3.54 (q, 4H), 6.78 (d, 1H), 7.62 (dd, 1H), 8.08 (dd, 1H).

### EXAMPLE 168

### N,N-diethyl-6-(2-methyl-1-pyrrolidinyl)nicotinamide

The desired product was prepared by substituting 2-methylpyrrolidine for N,N-diethylamine in Example 167. Purification and salt formation provided the dihydrochloride salt. MS m/e 262.1 (M+H)⁺; ¹H NMR (DMSO-d₆) δ 1.13 (t, 6H), 1.19 (d, 3H), 1.71-1.82 (m, 1H), 1.98-2.18 (m, 3H), 3.26-3.49 (m, 5H), 3.59-3.69 (m, 1H), 4.23.4.33 (m, 1H), 6.96 (d, 1H), 7.82 (dd, 1H), 8.09 (dd, 1H).

### EXAMPLE 169

### 6-[3-(aminocarbonyl)-1-piperidinyl]-N,N-diethylnicotinamide

The desired product was prepared by substituting nipecotamide for N,N-diethylamine in Example 167. Purification and salt formation provided the dihydrochloride salt. MS (M+H)+m/e 305.2; ¹H NMR (DMSO-d₆) δ 1.12 (t, 6H), 1.40-1.54 (m, 1H), 1.60-1.78 (m, 2H), 1.87-1.98 (m, 1H), 2.32-2.43 (m, 1H), 2.98-3.17 (m, 2H), 3.28-3.41 (m, 4H), 4.12-4.32 (m, 2H), 6.90 (s, 1H), 7.09 (d, 1H), 7.38 (s, 1H), 7.69 (dd, 1H), 8.10 (d, 1H).

### EXAMPLE 170

### N-[3-(dimethylamino)propyl]-N,6-dimethylnicotinamide

The desired product was prepared by substituting N-[3-(dimethylamino)propyl]-N-methylamine for N,N-diethylamine in Example 1. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS (M+H)+m/e 236; ¹H NMR (DMSO-d₆) δ 1.83-2.14 (br m, 2H), 2.53 (s, 3H), 2.75-2.85 (br m, 6H), 2.90-3.02 (br m, 3H), 3.02-3.30 (br m, 4H), 7.36-7.42 (m, 1H), 7.73-7.88 (br m, 1H), 8.57 (br s, 1H).

### EXAMPLE 171

### N-[2-(diethylamino)ethyl]-N,6-dimethylnicotinamide

The desired product was prepared by substituting N-[3-(diethylamino)ethyl]-N-methylamine for N,N-diethylamine in Example 1. After workup the crude compound was purified by HPLC on a C-18 column using a solvent system increasing in gradient from 5% to 100% acetonitrile/water containing 0.01% TFA over 50 minutes to provide the desired product as the trifluoroacetate salt. MS m/e 250 (M+H)⁺; ¹H NMR (DMSO-d₆) δ 1.25 (br t, 6H), 2.54 (s, 3H), 2.99 (s, 3H), 3.29 (br d, 6H), 3.79 (br s, 2H), 7.40 (d, 1H), 7.85 (d, 1H), 8.56 (s, 1H).

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A method of inhibiting angiogenesis comprising administering to a mammal in need of such treatment a therapeutically effective amount of a compound of formula (I) 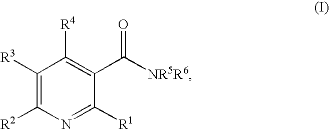
or a therapeutically salt thereof, wherein
R¹, R², R³, and R⁴ are independently selected from the group consisting of hydrogen, alkoxy, alkoxycarbonylalkyl, alkyl, amino, aryl, arylalkyl, cyano, cyanoalkyl, cycloalkyl, (cycloalkyl)alkyl, halo, haloalkoxy, haloalkyl, heteroaryl, heteroarylalkyl, heterocycle, (heterocycle)alkyl, hydroxy, hydroxyalkyl, and nitroalkyl; and
R⁵ and r⁶ are independently selected from the group consisting of hydrogen, alkoxyalkyl, alkyl, alkynyl, alkylsulfanylalkyl, aminoalkyl, cyanoalkyl, cycloalkyl, (cycloalkyl)alkyl, haloalkyl, heteroarylalkyl, and (heterocycle)alkyl.

2. The method of claim 1 wherein R⁴ is hydrogen.

3. The method of claim 2 wherein
R¹ and R² are hydrogen; and
R³ is other than hydrogen.

4. The method of claim 3 wherein the compound of formula (I) is selected from the group consisting of
N-ethyl-5-methylnicotinamide;
N,N-diethyl-5-(2-methylphenyl)nicotinamide;
methyl 4-{5-[(diethylamino)carbonyl]-3-pyridinyl}benzoate;
5-(3-aminophenyl)-N,N-diethylnicotinamide;
N,N-diethyl-5-(2-methoxyphenyl)nicotinamide;
N,N-diethyl-5-(4-methoxyphenyl)nicotinamide;
N,N-diethyl-5-(3-fluorophenyl)nicotinamide;
N,N-diethyl-5-(4-fluorophenyl)nicotinamide;
5-(3-chlorophenyl)-N,N-diethylnicotinamide;
5-(2-bromophenyl)-N,N-diethylnicotinamide;
5-(3-bromophenyl)-N,N-diethylnicotinamide;
5-(3-cyanophenyl)-N,N-diethylnicotinamide;
5-(4-acetylphenyl)-N,N-diethylnicotinamide;
5-(2,5-dimethylphenyl)-N,N-diethylnicotinamide;
5-(3,4-dimethylphenyl)-N,N-diethylnicotinamide;
5-(3,5-dimethylphenyl)-N,N-diethylnicotinamide;
5-(3-ethoxyphenyl)-N,N-diethylnicotinamide;
5-(2,4-dimethoxyphenyl)-N,N-diethylnicotinamide;
5-(2,5-dimethoxyphenyl)-N,N-diethylnicotinamide;
5-(3,4-dimethoxyphenyl)-N,N-diethylnicotinamide;
5-[3-(acetylamino)phenyl]-N,N-diethylnicotinamide;
N,N-diethyl-5-(3,4,5-trimethoxyphenyl)nicotinamide;
N,N-diethyl-3,4′-bipyridine-5-carboxamide; and
N,N-diethyl-5-(3-furyl)nicotinamide.

5. The method of claim 2 wherein
R¹ and R³ are hydrogen; and
R² is other than hydrogen.

6. The method of claim 5 wherein R⁵ and R⁶ are alkyl.

7. The method of claim 6 wherein the compound of formula (I) is selected from the group consisting of
N,N-diethyl-6-methylnicotinamide;
N,N-dimethyl-6-(1H-pyrazol-1-yl)nicotinamide;
N-butyl-N,6-dimethylnicotinamide;
N-isobutyl-N,6-dimethylnicotinamide;
N,6-dimethyl-N-pentylnicotinamide;
N,6-dimethyl-N-(3-methylbutyl)nicotinamide;
N-butyl-N-isopropyl-6-methylnicotinamide;
6-methyl-N,N-dipropylnicotinamide;
N-isopropyl-6-methyl-N-propylnicotinamide;
N-butyl-6-methyl-N-propylnicotinamide;
N-isopropyl-N,6-dimethylnicotinamide;
N,N-dibutyl-6-methylnicotinamide;
6-(4-aminophenyl)-N,N-diethylnicotinamide;
6-(3-acetylphenyl)-N,N-diethylnicotinamide;
6-[3-(acetylamino)phenyl]-N,N-diethylnicotinamide;
6-(3,5-dichlorophenyl)-N,N-diethylnicotinamide;
N,N-diethyl-6-(2-thienyl)nicotinamide;
6-bromo-N,N-diethylnicotinamide;
6-sec-butyl-N,N-diethylnicotinamide;
N,N-diethyl-6-(1-ethylpropyl)nicotinamide;
N,N-diethyl-6-hexylnicotinamide;
N,N-diethyl-6-(2-ethylbutyl)nicotinamide;
N,N-diethyl-6-(1-methylpentyl)nicotinamide;
N,N-diethyl-6-(1-ethylbutyl)nicotinamide;
6-(cyclohexylmethyl)-N,N-diethylnicotinamide;
6-(6-cyanohexyl)-N,N-diethylnicotinamide;
N,N-diethyl-6-(4-fluorobenzyl)nicotinamide;
methyl (3S)-3-{5-[(diethylamino)carbonyl]-2-pyridinyl}butanoate;
6-[(1S,2R,4R)-bicyclo [2.2.1]hept-2-yl]-N,N-diethylnicotinamide;
6-cyclohexyl-N,N-diethylnicotinamide;
6-(diethylamino)-N,N-diethylnicotinamide;
N,N-diethyl-6-(2-methyl-1-pyrrolidinyl)nicotinamide; and
6-[3-(aminocarbonyl)-1-piperidinyl]-N,N-diethylnicotinamide.

8. The method of claim 5 wherein one of R⁵ and R⁶ is hydrogen and the other is alkyl.

9. The method of claim 8 wherein the compound of formula (I) is selected from the group consisting of
N-ethyl-6-methylnicotinamide;
6-methyl-N-propylnicotinamide;
N-isopropyl-6-methylnicotinamide;
N-(sec-butyl)-6-methylnicotinamide;
N-isobutyl-6-methylnicotinamide;
N-(tert-butyl)-6-methylnicotinamide;
6-methyl-N-pentylnicotinamide;
6-methyl-N-(1-methylbutyl)nicotinamide;
6-methyl-N-(2-methylbutyl)nicotinamide;
6-methyl-N-(3-methylbutyl)nicotinamide;
6-methyl-N-neopentylnicotinamide; and
N-(3,3-dimethylbutyl)-6-methylnicotinamide.

10. The method of claim 5 wherein one of R⁵ and R⁶ is selected from the group consisting of hydrogen and alkyl and the other is selected from the group consisting of cycloalkyl and (cycloalkyl)alkyl.

11. The method of claim 10 wherein the compound of formula (I) is selected from the group consisting of
N-cyclohexyl-N,6-dimethylnicotinamide;
N-cyclopropyl-6-methylnicotinamide;
N-(cyclopropylmethyl)-6-methylnicotinamide;
N-cyclobutyl-6-methylnicotinamide;
N-cyclopentyl-6-methylnicotinamide;
N-(cyclopentylmethyl)-6-methylnicotinamide;
N-cyclohexyl-6-methylnicotinamide;
6-methyl-N-(2-methylcyclohexyl)nicotinamide;
6-methyl-N-(4-methylcyclohexyl)nicotinamide;
N-cycloheptyl-6-methylnicotinamide;
N-{[(1S,2R,5S)-6,6-dimethylbicyclo[3.1.1]hept-2-yl]methyl}-6-methylnicotinamide; and
N-(1-adamantylmethyl)-6-methylnicotinamide.

12. The method of claim 5 wherein one of R⁵ and R⁶ is selected from the group consisting of hydrogen and alkyl and the other is selected from the group consisting of hydrogen, alkoxyalkyl, cyanoalkyl, haloalkyl, and (heterocycle)alkyl.

13. The method of claim 12 wherein the compound of formula (I) is selected from the group consisting of
N-(cyanomethyl)-N,6-dimethylnicotinamide;
N-isopropyl-N-(2-methoxyethyl)-6-methylnicotinamide;
N-butyl-N-(cyanomethyl)-6-methylnicotinamide;
N,6-dimethyl-N-(tetrahydro-2-furanylmethyl)nicotinamide;
6-(2,2,2-trifluoroethoxy)nicotinamide;
6-methyl-N-(2,2,2-trifluoroethyl)nicotinamide;
N-(2-methoxyethyl)-6-methylnicotinamide;
N-(2-methoxy-1-methylethyl)-6-methylnicotinamide;
N-(2-ethoxyethyl)-6-methylnicotinamide;
N-(2-isopropoxyethyl)-6-methylnicotinamide;
6-methyl-N-(3-propoxypropyl)nicotinamide;
N-(3-methoxypropyl)-6-methylnicotinamide;
6-methyl-N-[(2S)-tetrahydro-2-furanylmethyl]nicotinamide;
6-methyl-N-[(2R)-tetrahydro-2-furanylmethyl]nicotinamide;
6-methyl-N-(tetrahydro-3-furanylmethyl)nicotinamide; and
N-(cyanomethyl)-6-methylnicotinamide.

14. The method of claim 5 wherein one of R⁵ and R⁶ is alkyl and the other is aminoalkyl.

15. The method of claim 14 wherein the compound of formula (I) is selected from the group consisting of
N-[2-(dimethylamino)ethyl]-N,6-dimethylnicotinamide;
N-[2-(dimethylamino)ethyl]-N-ethyl-6-methylnicotinamide;
N-[3-(dimethylamino)propyl]-N,6-dimethylnicotinamide; and
N-[2-(diethylamino)ethyl]-N,6-dimethylnicotinamide.

16. The method of claim 2 wherein
R¹ is as defined in claim 1; and
R² and R are hydrogen.

17. The method of claim 16 wherein the compound of formula (I) is selected from the group consisting of
N-ethyl-2-methylnicotinamide; and
N,N-diethylnicotinamide.

18. The method of claim 2 wherein
R¹ and R² are other than hydrogen; and
R³ is hydrogen.

19. The method of claim 18 wherein one of R⁵ and R⁶ is alkyl and the other is selected from the group consisting of hydrogen and alkyl.

20. The method of claim 19 wherein the compound of formula (I) is selected from the group consisting of
2-chloro-N-ethyl-N-isopropyl-6-methylnicotinamide;
2-chloro-N,N,6-trimethylnicotinamide;
N,N-diethyl-2-methyl-6-(trifluoromethyl)nicotinamide;
N-isopropyl-N,2-dimethyl-6-(trifluoromethyl)nicotinamide;
N-butyl-N,2-dimethyl-6-(trifluoromethyl)nicotinamide;
N-isobutyl-N,2-dimethyl-6-(trifluoromethyl)nicotinamide;
N-ethyl-2-methyl-N-propyl-6-(trifluoromethyl)nicotinamide;
N-butyl-N-isopropyl-2-methyl-6-(trifluoromethyl)nicotinamide;
N-isopropyl-2-methyl-N-propyl-6-(trifluoromethyl)nicotinamide;
N-butyl-2-methyl-N-propyl-6-(trifluoromethyl)nicotinamide;
N,N-dibutyl-2-methyl-6-(trifluoromethyl)nicotinamide;
N,N-diisobutyl-2-methyl-6-(trifluoromethyl)nicotinamide;
N-(sec-butyl)-2-chloro-6-methylnicotinamide;
2-chloro-6-methyl-N-pentylnicotinamide;
2-chloro-6-methyl-N-(2-methylbutyl)nicotinamide;
N-ethyl-2-methyl-6-(trifluoromethyl)nicotinamide;
2-methyl-N-propyl-6-(trifluoromethyl)nicotinamide;
N-isopropyl-2-methyl-6-(trifluoromethyl)nicotinamide;
N-butyl-2-methyl-6-(trifluoromethyl)nicotinamide;
N-(sec-butyl)-2-methyl-6-(trifluoromethyl)nicotinamide;
N-isobutyl-2-methyl-6-(trifluoromethyl)nicotinamide;
N-(tert-butyl)-2-methyl-6-(trifluoromethyl)nicotinamide;
2-methyl-N-pentyl-6-(trifluoromethyl)nicotinamide;
2-methyl-N-(1-methylbutyl)-6-(trifluoromethyl)nicotinamide;
2-methyl-N-(2-methylbutyl)-6-(trifluoromethyl)nicotinamide;
2-methyl-N-(3-methylbutyl)-6-(trifluoromethyl)nicotinamide;
N-(1,1-dimethylpropyl)-2-methyl-6-(trifluoromethyl)nicotinamide;
N-(]-ethylpropyl)-2-methyl-6-(trifluoromethyl)nicotinamide;
N-hexyl-2-methyl-6-(trifluoromethyl)nicotinamide; and
N-(3,3-dimethylbutyl)-2-methyl-6-(trifluoromethyl)nicotinamide.

21. The method of claim 18 wherein one of R⁵ and R⁶ is selected from the group consisting of hydrogen and alkyl and the other is selected from the group consisting of alkoxyalkyl, cyanoalkyl and cycloalkyl.

22. The method of claim 21 wherein the compound of formula (I) is selected from the group consisting of
2-chloro-N-cyclohexyl-N-ethyl-6-methylnicotinamide;
N-cyclohexyl-N,2-dimethyl-6-(trifluoromethyl)nicotinamide;
N-cyclohexyl-N-ethyl-2-methyl-6-(trifluoromethyl)nicotinamide;
N-(cyanomethyl)-N,2-dimethyl-6-(trifluoromethyl)nicotinamide;
N-(2-cyanoethyl)-N,2-dimethyl-6-(trifluoromethyl)nicotinamide;
N-butyl-N-(cyanomethyl)-2-methyl-6-(trifluoromethyl)nicotinamide;
2-chloro-N-(2-ethoxyethyl)-6-methylnicotinamide;
2-chloro-6-methyl-N-(3-propoxypropyl)nicotinamide;
2-chloro-N-(3-methoxypropyl)-6-methylnicotinamide;
2-chloro-N-(cyanomethyl)-6-methylnicotinamide;
2-chloro-N-cyclopropyl-6-methylnicotinamide;
2-chloro-N-cyclohexyl-6-methylnicotinamide;
2-chloro-6-methyl-N-(3-methylcyclohexyl)nicotinamide;
N-(4-tert-butylcyclohexyl)-2-chloro-6-methylnicotinamide;
2-chloro-N-cycloheptyl-6-methylnicotinamide;
N-(2-methoxy-1-methylethyl)-2-methyl-6-(trifluoromethyl)nicotinamide;
N-(2-isopropoxyethyl)-2-methyl-6-(trifluoromethyl)nicotinamide;
2-methyl-N-(3-propoxypropyl)-6-(trifluoromethyl)nicotinamide;
N-(3-methoxypropyl)-2-methyl-6-(trifluoromethyl)nicotinamide;
N-(cyanomethyl)-2-methyl-6-(trifluoromethyl)nicotinamide;
N-cyclobutyl-2-methyl-6-(trifluoromethyl)nicotinamide;
N-cyclopentyl-2-methyl-6-(trifluoromethyl)nicotinamide;
N-cyclohexyl-2-methyl-6-(trifluoromethyl)nicotinamide;
2-methyl-N-(2-methylcyclohexyl)-6-(trifluoromethyl)nicotinamide;
2-methyl-N-(4-methylcyclohexyl)-6-(trifluoromethyl)nicotinamide; and
N-2-adamantyl-2-methyl-6-(trifluoromethyl)nicotinamide.

23. The method of claim 18 wherein one of R⁵ and R⁶ is selected from the group consisting of hydrogen and alkyl and the other is selected from the group consisting of alkylsulfanylalkyl, alkynyl, (cycloalkyl)alkyl, and (heterocycle)alkyl.

24. The method of claim 23 wherein the compound of formula (I) is selected from the group consisting of
N-(1,3-dioxolan-2-ylmethyl)-N,2-dimethyl-6-(trifluoromethyl)nicotinamide;
N,2-dimethyl-N-2-propynyl-6-(trifluoromethyl)nicotinamide;
2-chloro-6-methyl-N-[(2S)-tetrahydro-2-furanylmethyl]nicotinamide;
2-chloro-6-methyl-N-[(2R)-tetrahydro-2-furanylmethyl]nicotinamide;
2-chloro-N-(cyclopropylmethyl)-6-methylnicotinamide;
2-chloro-N-(cyclohexylmethyl)-6-methylnicotinamide;
2-methyl-N-[2-(methylsulfanyl)ethyl]-6-(trifluoromethyl)nicotinamide;
2-methyl-N-[(2S)-tetrahydro-2-furanylmethyl]-6-(trifluoromethyl)nicotinamide;
2-methyl-N-[(2R)-tetrahydro-2-furanylmethyl]-6-(trifluoromethyl)nicotinamide;
2-methyl-N-2-propynyl-6-(trifluoromethyl)nicotinamide;
N-(cyclopropylmethyl)-2-methyl-6-(trifluoromethyl)nicotinamide; and
N-(1-adamantylmethyl)-2-methyl-6-(trifluoromethyl)nicotinamide.

25. A method of inhibiting angiogenesis comprising administering to a human in need of such treatment a therapeutically effective amount of a compound of claim 1 or a therapeutically acceptable salt thereof.

26. A method of treating cancer comprising administering to a mammal in need of such treatment a therapeutically effective amount of a compound of claim 1 or a therapeutically acceptable salt thereof.

27. A method of treating cancer comprising administering to a human in need of such treatment a therapeutically effective amount of a compound of claim 1 or a therapeutically acceptable salt thereof.

28. A compound of formula (II) 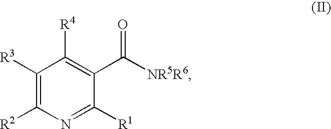
or a therapeutically acceptable salt thereof, wherein
R¹ and R⁴ are independently selected from the group consisting of hydrogen, alkoxy, alkoxycarbonylalkyl, alkyl, arylalkyl, cyano, cyanoalkyl, cycloalkyl, (cycloalkyl)alkyl, halo, haloalkoxy, haloalkyl, heteroaryl, heteroarylalkyl, heterocycle, (heterocycle)alkyl, hydroxy, hydroxyalkyl, and nitroalkyl;
R² and R³ are independently selected from the group consisting of hydrogen, alkoxy, alkoxycarbonylalkyl, alkyl, aryl, arylalkyl, cyano, cyanoalkyl, cycloalkyl, (cycloalkyl)alkyl, halo, haloalkoxy, haloalkyl, heteroaryl, heteroarylalkyl, heterocycle, (heterocycle)alkyl, hydroxy, and hydroxyalkyl; provided that at least one of R¹, R², R³, and R⁴ is other than hydrogen; and
one of R⁵ and R⁶ is alkyl and the other is selected from the group consisting of alkoxyalkyl and dialkylaminoalkyl.

29. The compound of claim 28 selected from the group consisting of
N-isopropyl-N-(2-methoxyethyl)-6-methylnicotinamide;
N-[2-(dimethylamino)ethyl]-N,6-dimethylnicotinamide;
N-[2-(dimethylamino)ethyl]-N-ethyl-6-methylnicotinamide;
N-[3-(dimethylamino)propyl]-N,6-dimethylnicotinamide; and
N-[2-(diethylamino)ethyl]-N,6-dimethylnicotinamide.

30. A pharmaceutical composition comprising a compound of claim 28 or a therapeutically acceptable salt thereof in combination with a therapeutically acceptable carrier or a therapeutically acceptable salt thereof.

29. A method of inhibiting angiogenesis comprising administering to a mammal in need of such treatment a therapeutically effective amount of a compound of claim 28 or a therapeutically acceptable salt thereof.

30. A method of inhibiting angiogenesis comprising administering to a human in need of such treatment a therapeutically effective amount of a compound of claim 28 or a therapeutically acceptable salt thereof.

31. A method of treating cancer comprising administering to a mammal in need of such treatment a therapeutically effective amount of a compound of claim 28 or a therapeutically acceptable salt thereof.

32. A method of treating cancer comprising administering to a human in need of such treatment a therapeutically effective amount of a compound of claim 28 or a therapeutically acceptable salt thereof.
